(19) United States
(12) Patent Application Publication
(10) Pub. No.: US 2026/0007203 A1
(43) Pub. Date: Jan. 8, 2026

(54) ARTICLES OF FOOTWEAR HAVING THERAPEUTIC ASSEMBLIES

(71) Applicant: Therabody, Inc., Los Angeles, CA (US)

(72) Inventors: Jaime Sanchez Solana, Los Angeles, CA (US); Eduardo Merino, Los Angeles, CA (US); Washington Alexander Silva Garces, Los Angeles, CA (US); Timothy Roberts, Sarasota, FL (US); Babak Baravarian, Santa Monica, CA (US); Benjamin Nazarian, Beverly Hills, CA (US); Jamie Dachisen, Santa Monica, CA (US)

(21) Appl. No.: 19/272,856

(22) Filed: Jul. 17, 2025

Related U.S. Application Data
(63) Continuation-in-part of application No. 18/737,898, filed on Jun. 7, 2024, now Pat. No. 12,402,686.
(60) Provisional application No. 63/508,249, filed on Jun. 14, 2023.

(51) Int. Cl. A43B 7/02; A43B 3/36; A43B 7/00; A43B 23/02; A61F 7/00

(52) U.S. Cl. CPC A43B 7/02; A43B 3/36; A43B 7/005; A43B 23/0205; A61F 7/00; A61F 2007/0045; A61F 2007/0093

(57) ABSTRACT

An article of footwear for warm-up and active recovery includes a sole and a light therapy system coupled to the sole. The light therapy system includes a plurality of LEDs, The article of footwear also includes a battery configured to power the light therapy system and control circuitry configured to control the light therapy system. The article of footwear may also include a vibration therapy system that includes a plurality of vibrating motors. The battery may power the vibration therapy system and the control circuitry may power the vibration therapy system.

### PRIORITY CLAIM

This application is a continuation-in-part of U.S. patent application Ser. No. 18/737,898, filed Jun. 7, 2024, entitled “ARTICLES OF FOOTWEAR HAVING THERAPEUTIC ASSEMBLIES;” and claims the benefit of U.S. Provisional Patent Application No. 63/508,249, filed Jun. 14, 2023, entitled “ARTICLES OF FOOTWEAR HAVING A VIBRATION ASSEMBLY.” The entireties of the disclosures of these applications are hereby incorporated herein by this reference.

### FIELD OF THE INVENTION

This disclosure relates to articles of footwear, such as slides, that include one or more therapeutic assemblies, and more particularly to articles of footwear that provide targeted vibration and/or targeted light therapy to areas of a user's foot.

### BACKGROUND OF THE INVENTION

Athletes and other individuals sometimes desire to use massage and therapeutic devices to warm up their muscles before working out and/or to recover after working out. Generic massage and therapeutic devices may not be ideal for a particular body part or muscle. Moreover, it can be time consuming to use a massage and therapeutic device for multiple body parts or muscles.

### SUMMARY OF THE INVENTION

An article of footwear for warm-up, active recovery, and treatment of pain is provided according to some embodiments. The article of footwear may include a sole and a light therapy system coupled to the sole. In some embodiments, the light therapy system includes a plurality of LEDs. In some embodiments, the article of footwear includes a battery to power the light therapy system and control circuity configured to control the light therapy system.

In some embodiments, the article of footwear also includes a transparent layer disposed above the sole. In some embodiments, the plurality of LEDs is disposed between the transparent layer and the sole. In some embodiments, the article of footwear also includes a layer disposed above the sole. In some embodiments, the layer defines holes. In some embodiments, the article of footwear includes a transparent cover disposed in each hole. In some embodiments, each of the plurality of LEDs is aligned with one of the transparent covers. In some embodiments, the plurality of LEDS is arranged in a plurality of rows.

In some embodiments, the article of footwear also includes a mounting layer disposed above the sole and a plurality of vibrating motors coupled to the mounting layer. In some embodiments, the battery is configured to power the plurality of vibrating motors and the control circuitry is configured to control the plurality of vibrating motors. In some embodiments, the mounting layer includes pockets. In some embodiments, each pocket is configured to receive a motor and each of the plurality of motors is disposed in one of the pockets. In some embodiments, the plurality of LEDs is disposed between the mounting layer and the sole. In some embodiments, the mounting layer defines a plurality of holes. In some embodiments, each hole is aligned with one of the plurality of LEDs. In some embodiments, a bottom surface of the mounting layer defines a cavity configured to receive the plurality of LEDs.

In some embodiments, the article of footwear also includes a bracket disposed above the sole. In some embodiments, the bracket is configured to support the plurality of LEDs. In some embodiments, the bracket includes a longitudinal section and a plurality of transverse sections. In some embodiments, the battery is configured to be recharged with a wireless charging device.

An article of footwear for warm-up and active recovery is provided according to some embodiments. The article of footwear may include a sole, a vibration system coupled to the sole, a light therapy system coupled to the sole, a battery to power the vibration system and the light therapy system, and control circuity to control the vibration system and the light therapy system. In some embodiments, the vibration system includes a plurality of motors. In some embodiments, the light therapy system includes a plurality of LEDs.

In some embodiments, the article of footwear also includes a mounting layer disposed above the sole. In some embodiments, the mounting layer has pockets, and each pocket can receive a motor. In some embodiments, each of the plurality of motors is disposed in one of the pockets and is configured to vibrate. In some embodiments, the mounting layer defines a plurality of holes. In some embodiments, each hole is aligned with one of the plurality of LEDs. In some embodiments, the plurality of LEDs is disposed between the mounting layer and the sole. In some embodiments, a bottom surface of the mounting layer defines a cavity configured to receive the plurality of LEDs. In some embodiments, the battery and the control circuitry are part of a control assembly and are disposed in a protective housing. In some embodiments, a bottom surface of the mounting layer defines a cavity configured to receive at least a portion of the control assembly.

In some embodiments, the article of footwear also includes a bracket disposed above the sole and configured to support the plurality of LEDs. In some embodiments, the bracket includes a longitudinal section and a plurality of transverse sections. In some embodiments, the article of footwear also includes a plurality of transparent covers. In some embodiments, each transparent cover is disposed within one of the plurality of holes of the mounting layer. In some embodiments, the plurality of LEDs is disposed between the bracket and the plurality of transparent covers. In some embodiments, the battery is configured to be recharged with a wireless charging device. In some embodiments, the article of footwear is a slide. In some embodiments, the plurality of motors includes a first motor disposed in a forefoot region of the article of footwear. In some embodiments, the plurality of motors includes a second motor disposed in a midfoot region of the article of footwear. In some embodiments, the plurality of motors includes a third motor disposed between the midfoot region and a rearfoot region of the article of footwear.

An article of footwear is provided according to some embodiments. The article of footwear may include a sole, a mounting layer coupled with the sole, a plurality of motors, a protective housing coupled to the mounting layer that contains a battery and control circuitry, and a switch coupled to the control circuitry and configured to turn the motors on and off. In some embodiments, the mounting layer has pockets with each pocket being configured to receive a motor. In some embodiments, each motor is disposed in one of the pockets and is configured to vibrate. In some embodiments, the battery is configured to power the motors and the control circuitry is configured to control the motors.

In some embodiments, the article of footwear is a slide. In some embodiments, the article of footwear also includes one or more cables coupling the control circuitry and the plurality of motors. In some embodiments, the plurality of motors are eccentric rotating mass vibrating motors. In some embodiments, the plurality of motors includes five motors. In some embodiments, the plurality of motors includes at least one motor disposed in a forefoot region of the sole, at least two motors disposed in a midfoot region of the sole, and at least two motors disposed in a rearfoot region of the sole.

In some embodiments, the pockets are disposed on a bottom of the mounting layer. In some embodiments, the pockets include five pockets. In some embodiments, the mounting layer is silicone. In some embodiments, the switch includes a button. In some embodiments, the button is disposed on an interior of the article of footwear. In some embodiments, the button is disposed such that a user's foot can operate the switch while wearing the article of footwear.

In some embodiments, the switch is configured to transition between operating modes of the motors. In some embodiments, the operating modes includes different speeds of vibration. In some embodiments, the operating modes include different patterns of vibration. In some embodiments, the article of footwear also includes a charging port configured to receive a charger to charge the battery. In some embodiments, the protective housing is disposed in a foot arch section of the sole. In some embodiments, the battery is rechargeable. In some embodiments, the one or more cables includes three sets of cables. In some embodiments, the article of footwear also includes a strap coupled to the sole. In some embodiments, the strap extends from a lateral side of the sole to a medial side of the sole such that an area between the strap and the sole is configured to receive a wearer's foot. In some embodiments, the article of footwear is injection molded.

In some embodiments, a top surface of the sole comprises cavities. In some embodiments, the cavities include a cavity for each of the plurality of motors. In some embodiments, the cavities include a cavity for the protective housing.

An article of footwear for warm-up and active recovery is provided according to some embodiments. The article of footwear may include a sole and a vibration system coupled to the sole. In some embodiments, the vibration system includes a plurality of vibrating motors, a battery, control circuitry, and a switch. In some embodiments, the vibration system includes three vibration zones.

In some embodiments, the three vibration zones include a front vibration zone having at least one vibrating motor of the plurality of vibrating motors disposed in a forefoot region of the sole. In some embodiments, the three vibration zones include a middle vibration zone having at least two vibrating motors of the plurality of vibrating motors disposed in a midfoot region of the sole. In some embodiments, the three vibration zones include a back vibration zone having at least two vibrating motors of the plurality of vibrating motors disposed in a rearfoot region of the sole. In some embodiments, the plurality of vibrating motors is at least partially embedded within the sole. In some embodiments, the article of footwear also includes a mounting layer coupled to the sole. In some embodiments, the mounting layer includes a plurality of pockets. In some embodiments, each of the plurality of vibrating motors is disposed in one of the plurality of pockets.

A slide is provided according to some embodiments. The slide may include a sole, a strap coupled to the sole that extends from a lateral side of the sole to a medial side of the sole such that an area between the strap and the sole is configured to receive a wearer's foot, and a vibration system that is coupled to the sole. In some embodiments, the vibration system includes a plurality of vibrating motors, a battery, control circuitry, and a switch.

The foregoing and other aspects, features, and advantages will be apparent from the DESCRIPTION and DRAWINGS, and from the CLAIMS.

### BRIEF DESCRIPTION OF THE DRAWINGS

Preferred and alternative examples of the present invention are described in detail below with reference to the following drawings.

Implementations will hereinafter be described in conjunction with the appended and/or included DRAWINGS.

FIG. 1 shows a side perspective view of an article of footwear having a vibration assembly according to some embodiments.

FIG. 2 shows a top schematic view of an article of footwear having a vibration assembly according to some embodiments.

FIG. 3 shows a schematic of disassembled components of an article of footwear having a vibration assembly according to some embodiments.

FIG. 4 shows a schematic of cabling for an article of footwear having a vibration assembly according to some embodiments.

FIG. 5 shows a top view of an article of footwear (with the vibration assembly removed) according to some embodiments.

FIGS. 6A-6C show potential areas of a user's foot that can be targeted for vibration therapy according to some embodiments.

FIG. 7A shows a top view of an article of footwear having therapeutic assemblies according to some embodiments.

FIG. 7B shows a top view of an article of footwear having therapeutic assemblies according to some embodiments.

FIG. 7C shows a side view of an article of footwear having therapeutic assemblies according to some embodiments.

FIG. 8 shows a cross-section schematic view of an article of footwear having therapeutic assemblies according to some embodiments.

FIG. 9 shows a cross-section schematic view of an article of footwear having therapeutic assemblies according to some embodiments.

FIG. 10 shows a cross-section schematic view of an article of footwear having therapeutic assemblies according to some embodiments.

FIG. 11 shows a perspective schematic view of a mounting layer for an article of footwear having therapeutic assemblies according to some embodiments.

FIG. 12 shows a side schematic view of a mounting layer for an article of footwear having therapeutic assemblies according to some embodiments.

FIG. 13A shows a top perspective view of an article of footwear having therapeutic assemblies according to some embodiments.

FIG. 13B shows a side view of an article of footwear having therapeutic assemblies according to some embodiments.

FIG. 14 shows a side view of an article of footwear having therapeutic assemblies according to some embodiments.

FIG. 15 shows a top view of a sole for an article of footwear having therapeutic assemblies according to some embodiments.

FIG. 16 shows a bottom perspective view of a mounting layer for an article of footwear having therapeutic assemblies according to some embodiments.

FIG. 17 shows a bottom view of an article of footwear having therapeutic assemblies (with the sole removed) according to some embodiments.

FIG. 18 shows a top perspective view of an article of footwear having therapeutic assemblies (with a mounting layer removed) according to some embodiments.

FIG. 19 shows a top perspective view of an article of footwear having therapeutic assemblies (with a mounting layer and LED covers removed) according to some embodiments.

FIG. 20 shows a cross section view of an article of footwear having therapeutic assemblies according to some embodiments.

FIG. 21 shows a bottom perspective view of an article of footwear having therapeutic assemblies (with the sole and cover of a control assembly removed) according to some embodiments.

FIG. 22A shows a charging system for an article of footwear having therapeutic assemblies according to some embodiments.

FIG. 22B shows a charging system with an article of footwear having therapeutic assemblies according to some embodiments.

FIG. 23 shows a bottom perspective view of an internal portion of an article of footwear having therapeutic assemblies according to some embodiments.

FIG. 24A shows a schematic view of a far infrared radiation system according to some embodiments.

FIG. 24B shows a schematic view of a thermoelectric cooling system according to some embodiments.

FIG. 25A shows a schematic view of a sole of an article of footwear according to some embodiments.

FIG. 25B shows a schematic view of a sole of an article of footwear according to some embodiments.

FIG. 25C shows a schematic view of a sole of an article of footwear according to some embodiments.

FIG. 25D shows a schematic view of a sole of an article of footwear according to some embodiments.

FIG. 26 shows a perspective view of an article of footwear having therapeutic assemblies according to some embodiments.

FIG. 27 shows a perspective view of an article of footwear having therapeutic assemblies according to some embodiments.

FIG. 28A shows a perspective view of an article of footwear having therapeutic assemblies according to some embodiments.

FIG. 28B shows a schematic view of a sole of an article of footwear according to some embodiments.

FIG. 28C shows a side schematic view of a mounting layer for an article of footwear having therapeutic assemblies according to some embodiments.

FIG. 29A shows a perspective view of an article of footwear according to some embodiments.

FIG. 29B shows a schematic view of a sole of an article of footwear according to some embodiments.

FIG. 30A shows a perspective view of an article of footwear according to some embodiments.

FIG. 30B shows a schematic view of a sole of an article of footwear according to some embodiments.

FIG. 31A shows a perspective view of an article of footwear according to some embodiments.

FIG. 31B shows a perspective view of an article of footwear according to some embodiments.

FIG. 31C shows a perspective view of an article of footwear according to some embodiments.

FIG. 31D shows a perspective view of an article of footwear according to some embodiments.

FIG. 31E shows a perspective view of an article of footwear according to some embodiments.

FIG. 32A shows a schematic view of a sole of an article of footwear according to some embodiments.

FIG. 32B shows a schematic view of a sole of an article of footwear according to some embodiments.

### DETAILED DESCRIPTION OF THE PREFERRED EMBODIMENT

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. The term “plurality,” as used herein, means more than one.

Throughout the description and claims of this specification, the words “comprise” and “contain” and variations of the words, for example “comprising” and “comprises,” mean “including but not limited to,” and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The present disclosure relates to articles of footwear, such as slides, that include one or more therapeutic assemblies, and more particularly to articles of footwear that provide targeted vibration and/or targeted light therapy to areas of a user's foot. Athletes and other individuals sometimes desire to use massage and therapeutic devices to warm up their muscles before working out and/or to recover after working out. Generic massage and therapeutic devices may not be ideal for a particular body part or muscle. Moreover, it can be time consuming to use a massage and therapeutic device for multiple body parts or muscles. A massage and therapeutic device that is directed specifically to providing vibration therapy and/or light therapy to a user's foot is desirable, especially one that is incorporated into an article of footwear, such as slides.

In some embodiments, an article of footwear comprises one or more therapeutic assemblies. The one or more therapeutic assemblies may include a vibration assembly, a light therapy assembly, or both. The article of footwear may have any of the features described in U.S. Provisional Application No. 63/508,249, filed Jun. 14, 2023, which is hereby incorporated by reference in its entirety into the present disclosure. For example, the vibration assembly may include any of the features described in U.S. Provisional Application No. 63/508,249, filed Jun. 14, 2023. In some embodiments, the one or more therapeutic assemblies may share components (e.g., power source, control system, etc.).

In some embodiments, an article of footwear comprises a sole and a vibration assembly coupled to the sole. The vibration assembly may include one or more vibrating devices (e.g., vibrating motors), a battery, and control circuitry. The motors may be disposed in locations that facilitate targeted vibration to key areas of the foot, such as soft tissue structures, fascia, muscles, and regions of nerve innervation. In some embodiments, the article of footwear comprises a mounting layer for the motors, the battery, and/or the control circuitry. The mounting layer may be coupled with the sole. In some embodiments, the article of footwear comprises a switch. The switch may be configured to turn the vibrating motors on and off and/or to transition between different operating modes. In some embodiments, the article of footwear comprises a slide.

In some embodiments, an article of footwear comprises a sole and a light therapy assembly coupled to the sole. The light therapy assembly may include one or more light-emitting diodes (LEDs), a battery, and control circuitry. The LEDs may be disposed in locations that facilitate targeted light therapy to key areas of the foot, such as soft tissue structures, fascia, muscles, and regions of nerve innervation. In some embodiments, the article of footwear comprises a mounting layer (such as an insole and/or other supporting structure) for the LEDs, the battery, and/or the control circuitry. The mounting layer may be coupled with the sole. In some embodiments, the article of footwear comprises an insole configured to allow light from the LEDs to reach a wearer's foot (e.g., a transparent insole, an insole with holes aligned with the LEDs). In some embodiments, the article of footwear comprises a switch. The switch may be configured to turn the LEDs on and off and/or to transition between different operating modes. In some embodiments, the article of footwear comprises a slide.

In some embodiments, an article of footwear comprises both a vibration assembly (such as the one described above) and a light therapy assembly (such as the one described above). In some embodiments, the battery and the control circuitry may be used to control both therapeutic assemblies. In addition, the switch may be used to transition between various operating modes for both the vibration assembly and the light therapy assembly. In some embodiments, multiple switches may be used (e.g., one for each therapeutic assembly). Slides with vibration and/or light therapy and a built-in battery provide a portable, lightweight, and comfortable massage and therapeutic device that can provide targeted therapy to a wearer's feet. Such slides can be used for warm-up prior to working out and for active recovery after working out. Such slides can also be used for treatment of pain, inflammation, and joint stiffness.

In some embodiments, the sole is shaped to provide massage, myofascial release, and/or a rolling benefit to the wearer. In addition, the sole's shape, material, or density may provide energy absorption, cushioning, and/or pressure distribution that leads to a more comfortable article of footwear (e.g., resulting in less stress on a wearer's joints). Thus, the sole's shape, material, or density may contribute to the therapeutic nature of the article of footwear (e.g., therapeutic effects related to pain, stiffness, and recovery).

Embodiments of articles of footwear having one or more therapeutic assemblies are described below. Features that are discussed with respect to any embodiments below may also be used in conjunction with other embodiments, even if not expressly discussed with respect to other embodiments.

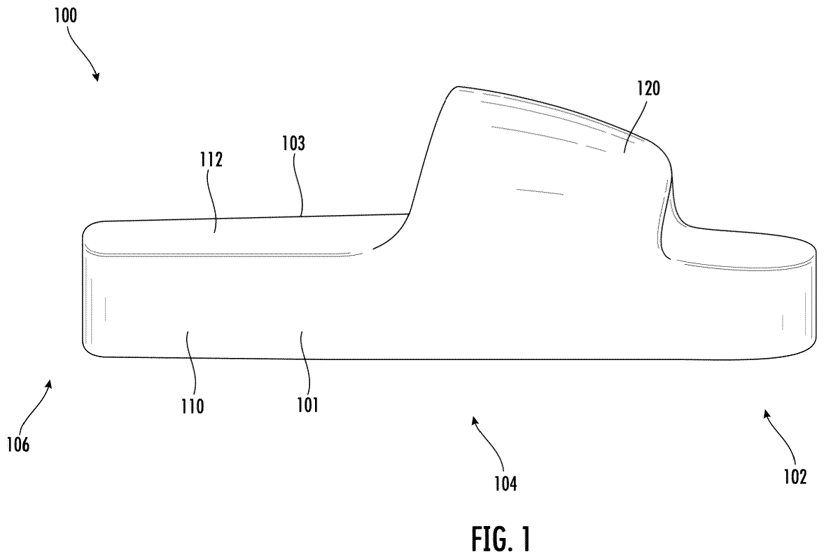

An article of footwear 100 according to some embodiments is shown, for example, in FIG. 1. Article of footwear 100 may have a forefoot region 102, a midfoot region 104, and a rearfoot region 106. Article of footwear 100 may have a lateral side 101 and a medial side 103. In some embodiments, article of footwear 100 comprises a sole 110. Article of footwear 100 may be a slide, as shown in FIG. 1. Thus, in addition to sole 110, article of footwear 100 may comprise a strap 120 coupled to sole 110. Strap 120 may be configured to extend from sole 110 at a lateral side 101 to sole 110 at a medial side 103 such that strap 120 and sole 110 together form an opening to receive a wearer's foot. In some embodiments, article of footwear 100 may be a different type of article of footwear, such as another type of sandal or a shoe with a sole and an upper section.

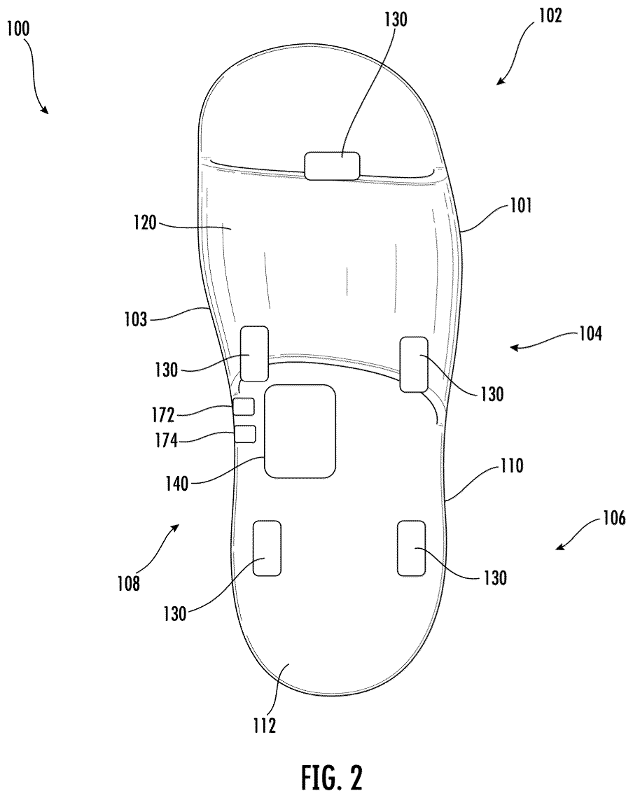

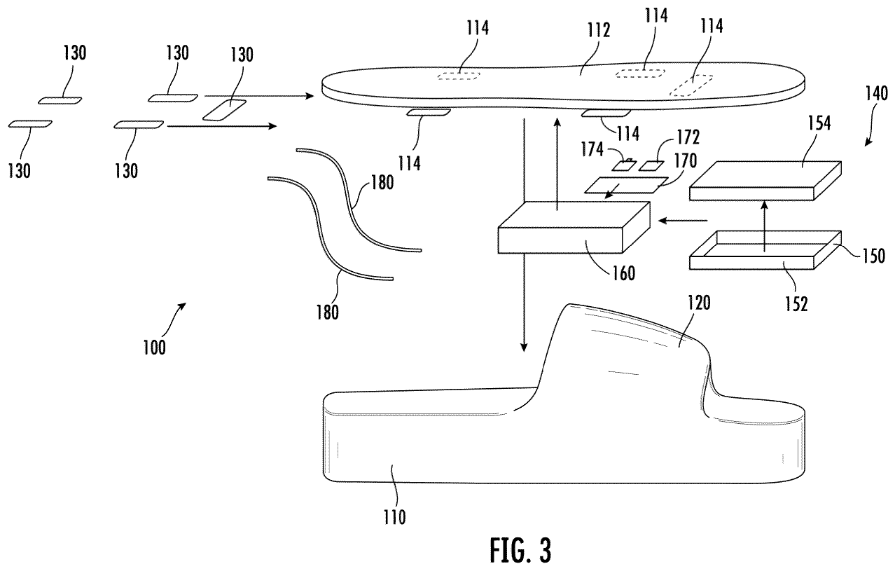

In some embodiments, article of footwear 100 comprises a vibration assembly. As shown, for example, in FIGS. 2-3, the vibration assembly may include one or more vibration devices 130 (e.g., motors 130) and a control assembly 140. The control assembly 140 may include a protective housing 150 containing a battery 160 and control circuitry 170 (see FIG. 3). In some embodiments, article of footwear 100 comprises a mounting layer 112 to hold one or more of the motors 130 and any of the components of the control assembly 140. These and other aspects of article of footwear 100 are discussed further below.

In some embodiments, motors 130 are vibrating motors. For example, the motors 130 may be eccentric rotating mass (ERM) vibrating motors. Other types of vibrating motors may also be used. The vibration of the motors 130 provides massage therapy to targeted areas of the wearer's foot. Local vibration has been shown to have both prophylactic and reactive benefits related to circulation, recovery, and pain. Furthermore, it has been associated with being able to help different medical conditions related to both circulatory and neurological disease states. The vibration devices 130 can include vibration frequencies of between 0-300 Hz and vibration amplitudes of between 0.5-12 mm.

In some embodiments, motors 130 are waterproof. In some embodiments, motors 130 are lightweight. For example, each motor 130 may be less than 10 grams. In some embodiments, each motor 130 may be less than 5 grams. Each motor 130 may be about 2 grams. In some embodiments, motors 130 may be designed to have minimum noise. In some embodiments, motors 130 may be pill-shaped motors. For example, motors 130 may have a diameter between 5 and 10 mm (e.g., 7 mm) and a length between 20 and 300 mm (e.g., 24.5 mm). In some embodiments, motors 130 with bigger dimensions may be used. Larger motors 130 may be beneficial in providing a stronger vibration. In some embodiments, motors 130 are coin-shaped instead of pill-shaped. Other shapes of motors 130 may also be used.

The number and placement of motors 130 may vary in different embodiments. In some embodiments, article of footwear 100 may include one to ten motors 130. For example, article of footwear 100 may include two, four, six, or eight motors. In some embodiments, article of footwear 100 includes more than ten motors 130. Motors 130 may be positioned under or adjacent to various key areas of the foot, such as soft tissue structures, fascia, muscles, and regions of nerve innervation. Target areas of the foot may include one or more of plantar fascia, lateral plantar fascia, flexor digitorum brevis tendons, flexor hallucis longus tendon, adductor hallucis, lumbricales, flexor hallucis brevis, flexor digiti minimi brevis, plantar interossei, flexor digitorum brevis, abductor hallucis, abductor digiti minimi, plantar aponeurosis, quadratus plantae, flexor digitorum longus tendons, deep peroneal nerve, superficial peroneal nerve, tibial nerve, sural nerve, medial plantar nerve, lateral plantar nerve, medial calcaneal nerve, saphenous nerve, or other structures.

As one example, FIG. 2 shows article of footwear 100 having five motors 130. FIG. 2 is drawn schematically to show approximate locations of motors 130 (and the other components of the vibration assembly) with respect to a plan view of article of footwear 100. Thus, these components would typically not be visible from a top view of article of footwear 100 but would instead be located underneath mounting layer 112 as discussed further below or otherwise embedded within sole 110. Motors 130 may include a motor 130 disposed in the forefoot region 102, two motors disposed in the midfoot region 104, and two motors 130 disposed in the rearfoot region 106.

Motors 130 may include a motor 130 disposed in the forefoot region 102, as shown in FIG. 2. For example, a motor 130 may be disposed in the forefoot region 102 such that it would be disposed underneath the ball of a wearer's foot when article of footwear 100 is worn. In some embodiments, this forefoot motor 130 may be disposed centrally between lateral side 101 and medial side 103. In some embodiments, this forefoot motor 130 may be disposed partially or entirely underneath strap 120. For example, FIG. 2 shows the forefoot motor 130 located such that a portion of the forefoot motor 130 is underneath strap 120 and a portion of the forefoot motor 130 extends past strap 120 towards a front of article of footwear 100. This forefoot motor 130 may be configured to provide massage therapy to the plantar fascia in the wearer's forefoot, superficial tendons and muscle in the forefoot (e.g., tendons of flexor digitorum brevis, flexor hallucis brevis), deep tendons and muscle in the forefoot (e.g., tendons of flexor digitorum longus, lumbricals, plantar interossei), and/or nerves in the forefoot (e.g., medial plantar nerve). Other locations of a forefoot motor 130 are also possible (e.g., to target a specific tendon, muscle, nerve, or other tissue structure). In some embodiments, multiple motors 130 may be disposed in forefoot region 102.

Motors 130 may include two motors 130 disposed in the midfoot region 104, as shown in FIG. 2. For example, a motor 130 may be disposed at a lateral side 101 of article of footwear 100 in the midfoot region 104 and another motor 130 may be disposed at a medial side 103 of article of footwear 100 in the midfoot region 104. The medial side motor 130 in the midfoot region 104 may be disposed such that it would be disposed underneath and/or adjacent to the arch of a wearer's foot when article of footwear 100 is worn. Motors 130 in the midfoot region 104 may be aligned with each other such that their positions in a longitudinal direction are lined up or they may be offset from each other. In some embodiments, the midfoot motors 130 may be disposed partially or entirely underneath strap 120. For example, FIG. 2 shows the midfoot motors 130 located such that a portion of the midfoot motors 130 are underneath strap 120 and a portion of the midfoot motors 130 extends past strap 120 towards a back of article of footwear 100. The medial midfoot motor 130 may be configured to provide massage therapy to the plantar fascia in the wearer's medial midfoot, superficial tendons and muscle in the medial midfoot (e.g., flexor hallucis brevis, abductor hallucis), deep tendons and muscle in the medial midfoot (e.g., tendons of tibialis posterior and flexor hallucis longus, flexor hallucis brevis), and/or nerves in the medial midfoot (e.g., medial plantar nerve, saphenous nerve). The lateral midfoot motor 130 may be configured to provide massage therapy to the plantar fascia in the wearer's lateral midfoot, superficial tendons and muscle in the lateral midfoot (e.g., flexor digiti minimi brevis, abductor digiti minimi), deep tendons and muscle in the lateral midfoot (e.g., tendons of fibularis brevis and fibularis longus), and/or nerves in the lateral midfoot (e.g., lateral plantar nerve, sural nerve). Other locations of midfoot motors 130 are also possible (e.g., to target a specific tendon, muscle, nerve, or other tissue structure). In some embodiments, a different number of motors 130 may be disposed in midfoot region 104 (such as only one motor 130 or more than two).

Motors 130 may include two motors 130 disposed in the rearfoot region 106, as shown in FIG. 2. For example, a motor 130 may be disposed at a lateral side 101 of article of footwear 100 in the rearfoot region 106 and another motor 130 may be disposed at a medial side 103 of article of footwear 100 in the rearfoot region 106. The medial side motor 130 in the rearfoot region 106 may be disposed such that it would be disposed underneath and/or adjacent to the heel of a wearer's foot when article of footwear 100 is worn. Motors 130 in the rearfoot region 106 may be aligned with each other such that their positions in a longitudinal direction are lined up or they may be offset from each other. The medial rearfoot motor 130 may be configured to provide massage therapy to the plantar fascia in the wearer's medial rearfoot, superficial tendons and muscle in the medial rearfoot (e.g., flexor hallucis brevis, abductor hallucis), deep tendons and muscle in the medial rearfoot (e.g., tendons of tibialis posterior and flexor hallucis longus, flexor hallucis brevis, quadrates plantae), and/or nerves in the medial rearfoot (e.g., tibial nerve, saphenous nerve). The lateral rearfoot motor 130 may be configured to provide massage therapy to the plantar fascia in the wearer's lateral rearfoot, superficial tendons and muscle in the lateral rearfoot (e.g., flexor digiti minimi brevis, abductor digiti minimi, plantar aponeurosis), deep tendons and muscle in the lateral rearfoot (e.g., tendons of fibularis brevis and fibularis longus), and/or nerves in the lateral rearfoot (e.g., tibial nerve, sural nerve). Other locations of rearfoot motors 130 are also possible (e.g., to target a specific tendon, muscle, nerve, or other tissue structure). In some embodiments, a different number of motors 130 may be disposed in rearfoot region 106 (such as only one motor 130 or more than two).

As already noted above, article of footwear 100 may have a different number of motors 130 and may use different locations for motors 130 other than those discussed above and shown in FIG. 2. In some embodiments, motors 130 may be placed to have different vibration zones. For example, the vibration assembly may provide two or more vibration zones. In some embodiments, the vibration zones may include a front vibration zone (e.g., a vibration zone provided by motor(s) 130 in forefoot region 102), a middle vibration zone (e.g., a vibration zone provided by motor(s) 130 in midfoot region 104), and a back vibration zone (e.g., a vibration zone provided by motor(s) 130 in rearfoot region 106). In some embodiments, control assembly 140 may be configured to allow each vibration zone to be turned on individually or together with one or more of the other vibration zones.

In some embodiments, motors 130 are coupled to mounting layer 112, as shown, for example, in FIG. 3. Mounting layer 112 may include one or more pockets 114. Each pocket 114 may be configured to receive a motor 130. Thus, the number of pockets 114 on mounting layer 112 may be the same as the number of motors 130 discussed above. For example, mounting layer 112 may include five pockets 114. In addition, the location of pockets 114 on mounting layer 112 and with respect to the rest of article of footwear 100 may be the same as the locations of motors 130 discussed above. In some embodiments, pockets 114 are disposed on a bottom of mounting layer 112. Other components of the vibration assembly may also be coupled to mounting layer 112. In some embodiments, the other components of the vibration assembly may be disposed underneath mounting layer 112 (e.g., embedded in a material of sole 110 or disposed in a cavity of sole 110 without any bonding connection to mounting layer 112).

Mounting layer 112 may be rigid enough to be durable but flexible to minimize noise of motors 130. Various thicknesses of mounting layer 112 may be used (e.g., depending on the material of mounting layer 112). Mounting layer 112 may be thin enough such that the vibration of motors 130 is felt by a wearer (so that the massage therapy is effective), while being thick enough to provide a reliable mounting structure for motors 130. In some embodiments, mounting layer 112 is made of silicone. Other materials may also be used for mounting layer 112 (e.g., rubber, foam, etc.). In some embodiments, mounting layer 112 provides a soft material to be in contact with the wearer's foot (e.g., silicone, rubber, foam, etc.). In some embodiments, mounting layer 112 may be a plastic structure to support motors 130 with a soft material covering a top of mounting layer 112 to provide a soft surface to be in contact with the wearer's foot. In some embodiments, mounting layer 112 is coupled to sole 110. The connection of mounting layer 112 to sole 110 may be any flexible, reliable, and waterproof connection. In some embodiments, mounting layer 112 is coupled to sole 110 using contact cement. In some embodiments, mounting layer 112 is glued to sole 110. In some embodiments, mounting layer 112 is stitched to sole 110. In some embodiments, mounting layer 112 is an additional layer that is added to sole 110. For example, sole 110 may provide a hollow center surrounded by external surfaces forming sole 110, and mounting layer 112 may be attached to sole 110 in the hollow center. In some embodiments, mounting layer 112 may be covered either from a top or bottom to enclose mounting layer 112 within sole 110.

In some embodiments, a top surface of mounting layer 112 forms a top surface of sole 110 on which a wearer's foot may rest. In some embodiments, a different material is disposed above mounting layer 112 to form a top surface of sole 110. In some embodiments, article of footwear 100 does not include a mounting layer 112. For example, the components of the vibration assembly (e.g., motors 130 and components of control assembly 140, etc.) may be embedded within a material of sole 110 without using a mounting layer 112.

Control assembly 140 is configured to power and control motors 130 to provide massage therapy. In some embodiments, control assembly 140 may be disposed in an arch region 108 of article of footwear 100 (i.e., a region that corresponds to an arch of the wearer's foot when article of footwear 100 is worn), as shown in FIG. 2. Control assembly 140 may include protective housing 150 containing a battery 160 and control circuitry 170 (see FIG. 3). Control assembly 140 may also include cabling 180 coupling control circuitry 170 to motors 130.

In some embodiments, control circuitry 170 is configured to control operation of motors 130. For example, control circuitry 170 may control the voltage provided to motors 130 to turn the motors on and off and/or to transition between different operating modes (vibration speeds, vibration patterns (e.g., vibration frequencies, intensities, or durations) vibration zones, etc.). In some embodiments, control circuitry 170 may also control the charging of battery 160. In some embodiments, control circuitry 170 may comprise a printed circuit board. Control circuitry 170 may be contained within protective housing 150.

In some embodiments, a charging port 172 is coupled to control circuitry 170. Charging port 172 may be configured to receive a charger to charge battery 160. In some embodiments, charging port 172 is a USB-C port. Other types of ports may also be used for charging port 172. Charging port 172 may be accessible from an exterior of article of footwear 100. For example, charging port 172 may be disposed on an exterior wall of medial side 103 of sole 110, such as at arch region 108 (see FIG. 2). In some embodiments, charging port 172 is supported and/or contained by protective housing 150.

In some embodiments, a switch 174 is coupled to control circuitry 170. Switch 174 is configured to turn motors 130 on and off. For example, a wearer may use switch 174 to turn motors 130 on and off. In some embodiments, switch 174 is also configured to transition between operating modes of motors 130. The operating modes may be different speeds of vibration, different patterns of vibration, or both. In some embodiments, the operating modes may include three different speeds. As one example, a first operation of the switch 174 may turn motors 130 on at a high speed, a second operation of switch 174 may change the motors to a medium speed, a third operation of switch 174 may change the motors to a low speed, and a fourth operation of switch 174 may turn the motors off. In addition, the operating modes may include different vibration zones. As one example, a user may first operate switch 174 to turn all motors 130 on, then to turn only a front vibration zone on (with the others off), then to turn only a middle vibration zone on (with the others off), then to turn only a back vibration zone on (with the others off), and then to turn all motors 130 off. Any other operating modes may also be used with the switch 174 allowing for a user to transition between any such operating modes. In some embodiments, multiple switches 174 may be included.

In some embodiments, switch 174 may be a button 174 (e.g., a button that is pushed to operate). In some embodiments, button 174 is disposed on an interior of article of footwear. For example, button 174 may be disposed such that a user's foot can operate button 174 while wearing article of footwear 100. Switch 174 may be located at medial side 103 in arch region 108 (see FIG. 2). Other locations may also be used. In some embodiments, switch 174 is supported and/or contained by protective housing 150. In some embodiments, switch 174 may include multiple buttons 174 located in different areas of the article of footwear 100.

In some embodiments, battery 160 is configured to power motors 130. In some embodiments, battery 160 is a rechargeable battery. For example, battery 160 may be a lithium-ion battery, such as a lithium-polymer battery. In some embodiments, battery 160 is configured to operate on a single charge for a run time of between one and six hours (e.g., two hours).

In some embodiments, protective housing 150 contains battery 160 and control circuitry 170. In some embodiments, protective housing 150 also contains charging port 172 and switch 174. Protective housing 150 may be made of plastic, such as polycarbonate, acrylonitrile butadiene styrene, polyamide, or some other plastic material. Any other waterproof material may also be used. In some embodiments, protective housing 150 comprises a lower section 152 and an upper section 154. Lower section 152 and upper section 154 may be joined together such that protective housing 150 is waterproof. For example, lower section 152 and upper section 154 may be joined using ultrasonic welding. In some embodiments, protective housing 150 may comprise connection points for cabling 180 to allow cabling 180 to electrically couple to control circuitry 170 inside protective housing 150.

In some embodiments, cabling 180 comprises one or more cables that couple the motors 130 to control circuitry 170 (and thus battery 160). In some embodiments, cabling 180 is waterproof. Cabling 180 may be connected to motors 130 and to control circuity 170 by being soldered.

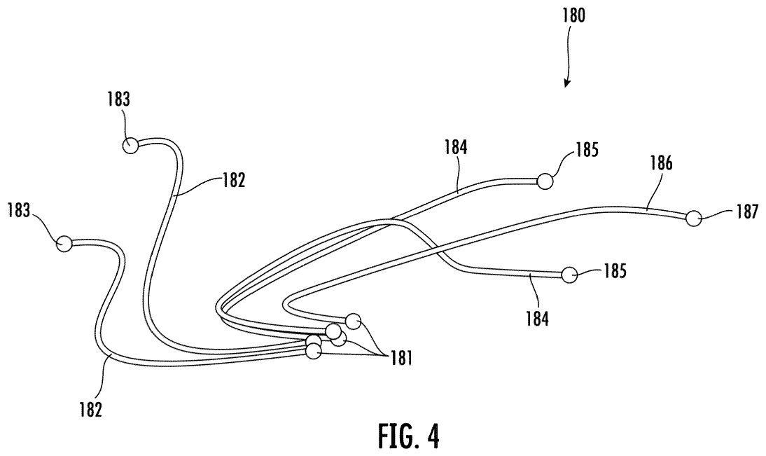

Cabling 180 may include three sets of cables, as shown, for example, in FIG. 4. The different sets of cables may correspond to vibration zones. Thus, a different number of sets of cables may also be used. In FIG. 4, the three sets of cables comprise a first set of two cables 182, a second set of two cables 184, and a third set of cables (having only one cable 186). Each cable 182, 184, 186 extends from connection points 181 at protective housing 150 and/or control circuitry 170 to respective connection points at motors 130. In some embodiments, the first set of cables 182 may correspond to a back vibration zone and thus be connected to rearfoot motors 130 at connection points 183. In some embodiments, the second set of cables 184 may correspond to a middle vibration zone and thus be connected to midfoot motors 130 at connection points 185. In some embodiments, cable 186 may correspond to a front vibration zone and thus be connected to forefoot motor 130 at connection point 187.

Sole 110 may provide the main structure for article of footwear 100. In some embodiments, the shape of sole 110 contributes to article of footwear 100 being a therapeutic device. For example, sole 110 may be shaped to provide massage to a wearer's foot while article of footwear 100 is being worn. Sole 110 may be shaped to facilitate myofascial release while a wearer is walking in article of footwear 100. For example, sole 110 may provide a rolling benefit. In some embodiments, a shape of an external surface of mounting layer 112 (e.g., the surface facing the wearer's foot) may be shaped to provide massage, myofascial release, and/or rolling benefits. The shape of the external surface of mounting layer 112 may also be shaped to provide support for different areas of a wearer's foot.

In some embodiments, sole 110 is configured to provide energy absorption, cushioning, and pressure distribution that increases the comfort for the wearer while using article of footwear 100. For example, a shape, material, and/or density of sole 110 may be selected that lead to better energy absorption, cushioning, and pressure distribution, thus leading to a more comfortable experience. In some embodiments, for example, sole 110 may provide additional support under the wearer's foot arch.

In some embodiments, sole 110 may be made of polyurethane or ethyl-vinyl acetate. In some embodiments, sole 110 may be made of a foam material. Strap 120 may be made of the same material as sole 110. Sole 110 may be made using a variety of manufacturing methods. In some embodiments, sole 110 is injection molded. In some embodiments, strap 120 and sole 110 are molded with a single injection. In some embodiments, strap 120 is manufactured separately from sole 110 and then the two may be joined together. In some embodiments, sole 110 and/or strap 120 are 3D printed. In some embodiments, sole 110 (or the external-facing side of mounting layer 112) may include a conductive material that is electrically grounded (e.g., by a wired connection to ground in control circuitry 170) to provide a grounding or earthing effect to the wearer of the article of footwear 100 when the wearer's foot is in contact with the conductive material. In some embodiments, the grounding effect provided by the conductive material may provide therapeutic benefits to the wearer, such as reducing inflammation and pain and promoting wound healing. In some embodiments, the conductive material may be located in any region of article of footwear 100, such as forefoot region 102, a midfoot region 104, and/or a rearfoot region 106.

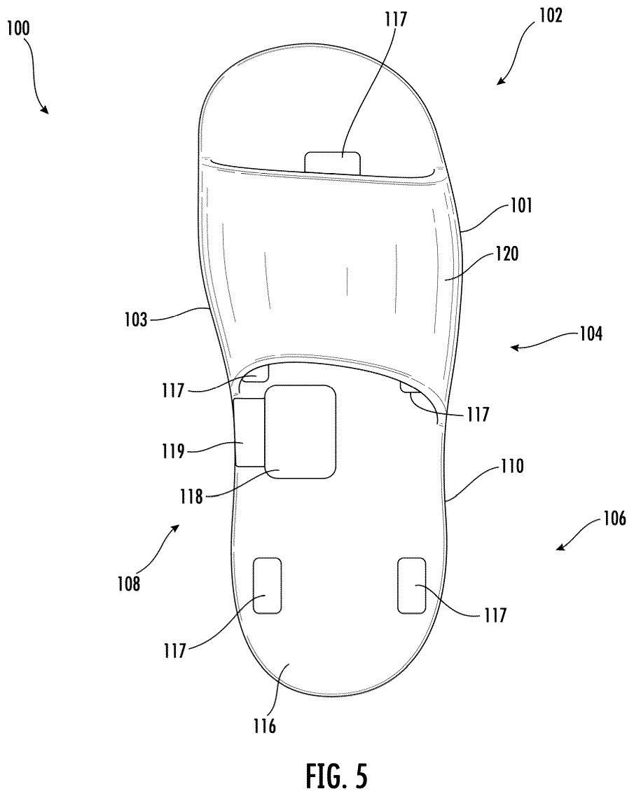

In some embodiments, sole 110 may be configured to accommodate components of the vibration system. For example, sole 110 may comprise a surface 116 (as shown in FIG. 5) that defines a plurality of cavities sized and shaped to receive components of the vibration system. Surface 116 may be a top surface of the material that makes up sole 110. Thus, the cavities may be formed during the injection molding process. In some embodiments, surface 116 defines motor cavities 117 configured to receive motors 130. For example, motors 130 may be disposed within pockets 114 of mounting layer 112 and the pockets 114 and motors 130 may be fully or partially disposed within motor cavities 117. Thus, the number of motor cavities in surface 116 of sole 110 may be the same as the number of motors 130 discussed above. For example, surface 116 may define five motor cavities 117. In addition, the location of motor cavities 117 on surface 116 may be the same as the locations of motors 130 discussed above.

In some embodiments, surface 116 defines protective housing cavity 118. Protective housing cavity 118 may be disposed in arch region 108 of sole 110. Protective housing cavity 118 is configured to receive protective housing 150. In some embodiments, surface 116 defines a cavity 119 configured to receive charging port 172 and switch 174. Cavity 119 may be disposed in arch region 108 of sole 110. For example, cavity 119 may be disposed adjacent to protective housing cavity 118.

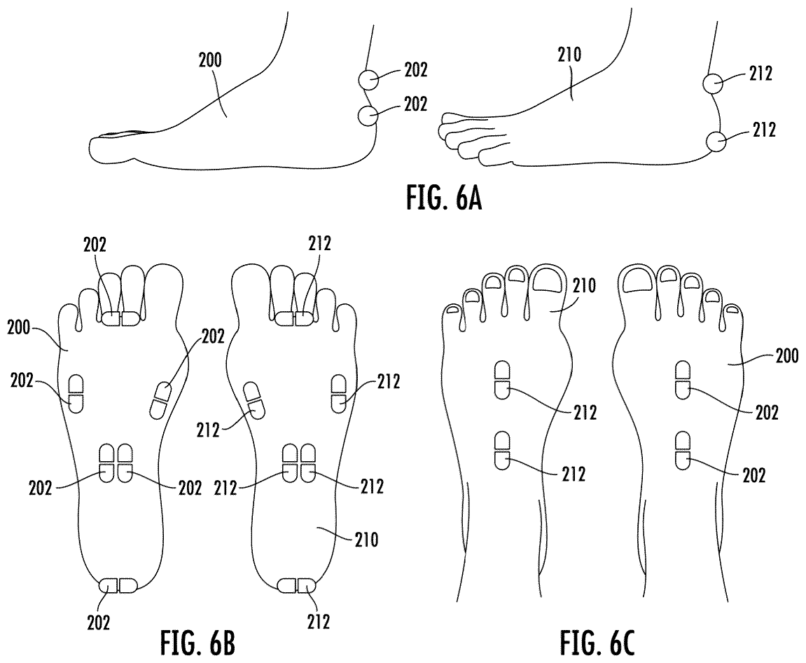

Many additional implementations are possible. For example, although FIGS. 1-3 and 5 show article of footwear 100 as a slide, other articles of footwear may also be used. In addition, although FIGS. 1-3 and 5 show particular number and locations of motors 130, other variations may be used. For example, FIGS. 6A-6C illustrate other potential areas of a wearer's feet that may be targeted with vibrating motors for massage therapy using slides or other articles of footwear. FIG. 6A shows target areas 202 on a right foot 200 of a wearer and target areas 212 on a left foot of a wearer in a side view. FIG. 6B shows target areas 202 on a right foot 200 of a wearer and target areas 212 on a left foot of a wearer in a bottom or plantar view. FIG. 6C shows target areas 202 on a right foot 200 of a wearer and target areas 212 on a left foot of a wearer in a top or dorsal view.

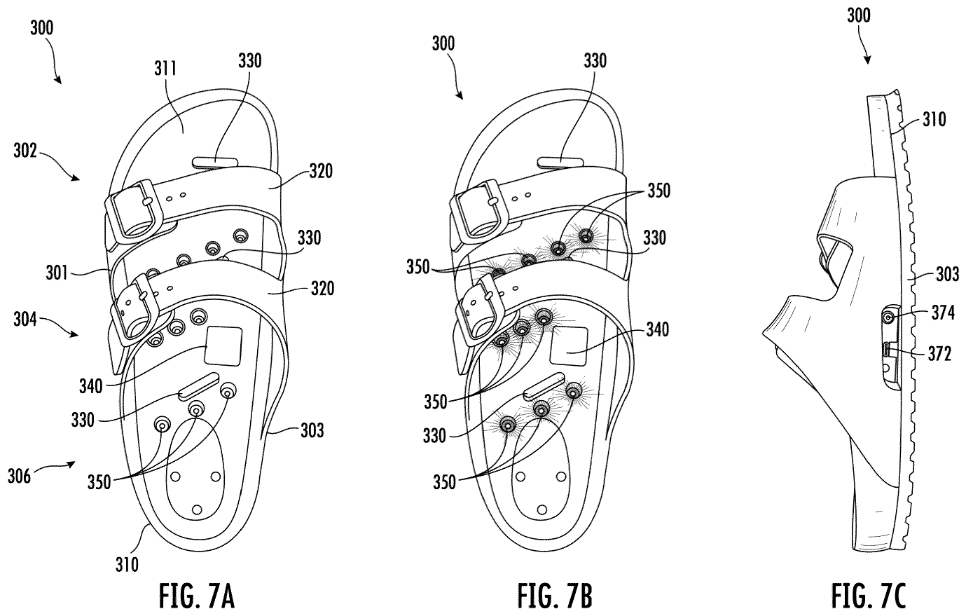

In some embodiments, an article of footwear may include a light therapy assembly in addition to or instead of a vibration assembly. For example, an article of footwear 300 according to some embodiments is shown in FIGS. 7A-7C having both a light therapy assembly and a vibration assembly. Features discussed with respect to article of footwear 100 above may also be implemented as part of article of footwear 300. Similarly, features discussed with respect to article of footwear 300 below may also be implemented as part of article of footwear 100. In addition, while article of footwear 300 comprises both a light therapy assembly and a vibration assembly, in some embodiments, an article of footwear comprises a light therapy assembly (such as the one discussed with respect to article of footwear 300) and does not include a vibration assembly.

Article of footwear 300 may have a forefoot region 302, a midfoot region 304, and a rearfoot region 306. Article of footwear 300 may have a lateral side 301 and a medial side 303. In some embodiments, article of footwear 300 comprises a sole 310. Article of footwear 300 may be a slide, as shown in FIGS. 7A-7C. Thus, in addition to sole 310, article of footwear 300 may comprise a strap 320 coupled to sole 310. In some embodiments, article of footwear 300 may include multiple straps 320 (e.g., two straps 320). Strap(s) 320 may be configured to extend from sole 310 at lateral side 301 to sole 310 at medial side 303 such that strap(s) 320 and sole 310 together form an opening to receive a wearer's foot. In some embodiments, each strap 320 may comprise two parts, one extending from lateral side 301 of sole 310 and one extending from medial side 303 of sole 310. The two parts may fasten together to form strap 320. For example, the two parts may fasten with a buckle system (see FIGS. 7A-7B), a hook and loop system, a button system, a snap system, or other fastening systems. In some embodiments, article of footwear 300 may be a different type of article of footwear, such as another type of sandal or a shoe with a sole and an upper section. In some embodiments, the article of footwear 300 may include a fastening system and/or fastening mechanism that allows the footwear to be secured onto a wearer's foot. In some embodiments, the shoe's fastening mechanism may allow the footwear 300 to be adjusted or tightened properly to ensure a better fit for the wearer's foot, and to ensure an efficient delivery of the therapeutic treatment (e.g., light therapy and/or vibration therapy) to the wearer's foot.

In some embodiments, article of footwear 300 comprises a vibration assembly, which may have the features discussed above with respect to article of footwear 100, and a light therapy assembly. FIG. 7A shows article of footwear 300 with light therapy assembly turned off while FIG. 7B shows article of footwear 300 with light therapy assembly turned on. In some embodiments, a top layer 311 of article of footwear 300 is transparent. For example, top layer 311 may be made of a transparent material (e.g., transparent silicone). Configuring top layer 311 to be transparent facilitates light therapy for a wearer's foot from devices within article of footwear 300, such as LEDs.

Referring again to FIG. 7A, article of footwear 300 comprises a control assembly 340. Control assembly 340 is disposed below top layer 311 (e.g., coupled to a mounting layer as discussed above). In some embodiments, control assembly 340 is visible through transparent top layer 311, as shown in FIGS. 7A and 7B. Control assembly 340 is configured to power and control the vibration assembly and the light therapy assembly, and control assembly 340 may include any of the features discussed above for control assembly 140. For example, control assembly 340 may be disposed in an arch region of article of footwear 300 (i.e., a region that corresponds to an arch of the wearer's foot when article of footwear 300 is worn).

In some embodiments, article of footwear 300 comprises one or more vibration devices 330 (e.g., motors 330). Motors 330 are disposed below top layer 311 (e.g., within a mounting layer as discussed above). In some embodiments, motors 330 are visible through transparent top layer 311, as shown in FIGS. 7A and 7B. In some embodiments, motors 330 may have any of the features discussed above for motors 130. In some embodiments, motors 330 have a diameter between 5 and 10 mm (e.g., 8 mm).

The number and placement of motors 330 may vary in different embodiments. In some embodiments, article of footwear comprises three motors 330. Motors 330 may include a first motor 330 in forefoot region 302 (which is shown just in front of forward strap 320 in FIGS. 7A and 7B), a second motor 330 in midfoot region 304 (which is partially shown just in front of the rearward strap 320 in FIGS. 7A and 7B), and a third motor 330 between midfoot region 304 and rearfoot region 306 (e.g., adjacent to and located rearward of control assembly 340). Each of motors 330 may be disposed anywhere between medial side 303 and lateral side 301 of article of footwear 300 (e.g., centrally, closer to medial side 303, or closer to lateral side 301). Motors 330 may be positioned to provide targeted vibration therapy to certain portions of the wearer's foot (as discussed above).

In some embodiments, article of footwear 300 comprises one or more light therapy devices 350 (e.g., light-emitting diodes (LEDs) 350). In some embodiments, article of footwear comprises between two and thirty LEDs 350, between ten and twenty LEDs 350, or between twelve and sixteen LEDs 350 (e.g., 14 LEDs 350). Different amounts of LEDs 350 (e.g., more than 30) may be used in some embodiments. The number and placement of LEDs 350 may vary in different embodiments.

LEDs 350 may provide light therapy to help heal, increase circulation, stimulate metabolic processes, regenerate muscles, tendons, bones, joints, and other tissue, thus contributing to recovery after exercise and the treatment of both acute and chronic conditions associated with pain and inflammation. Red LEDs with visible red light in the spectrum of 620-700 nm and infrared LEDs producing near infrared (NIR) of wavelengths 700-2500 nm can be used to provide specific therapeutic fluence (dose in J/cm2). The therapeutic fluence is the product of a power density and treatment time that is both safe and effective in producing the physiological and neurological responses that produce the therapeutic benefit. In some embodiments, the power density of visible red light will be <300 mW/cm2 with the power density of NIR <750 mW/cm2. In some embodiments, the fluence provided to the tissues will range between 20-1000 J/cm2 depending on purpose of use and intended outcomes. Visible red light can be used for therapeutic benefits and as visual feedback to the user (e.g., wearer of the article of footwear 300) to show that the device is working and producing light. LEDs 350 may be disposed in an array across article of footwear 300. In some embodiments, LEDs 350 are arranged in a number of rows (e.g., one to six rows, two to four rows, six to ten rows, etc.), with each row having two to eight LEDs 350 (e.g., three to four LEDs). Each row of LEDs 350 may span across the width of article of footwear 300. In some embodiments, LEDs 350 of each row are spaced equally apart from each other.

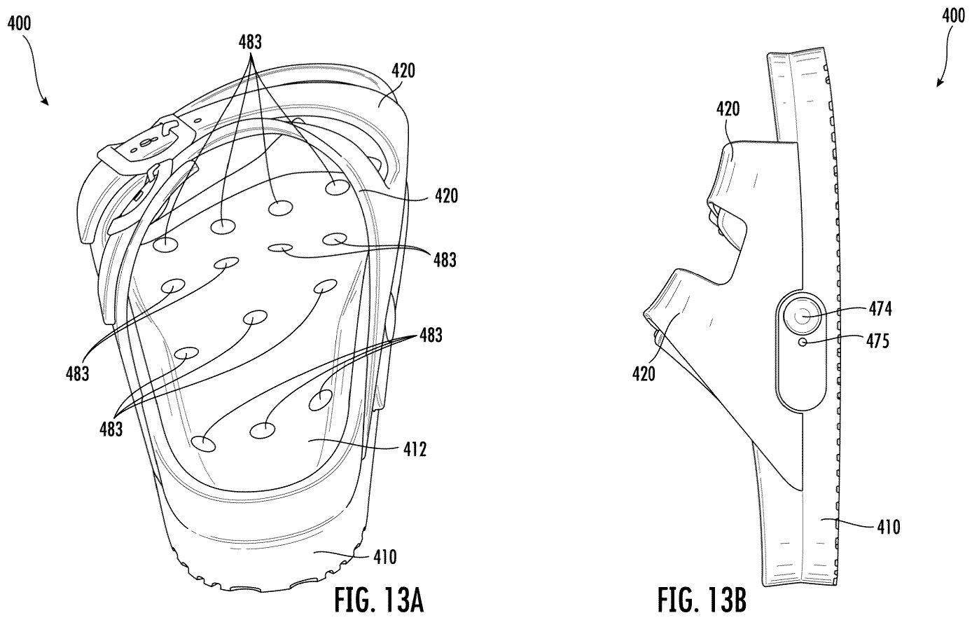

In some embodiments, LEDs 350 are arranged in four rows. The foremost row (e.g., the row disposed in or closest to forefoot region 302) may be disposed below forward strap 320 (similar to what is shown in FIG. 13A). The foremost row may include four LEDs 350. In some embodiments, the foremost row of LEDs 350 is disposed at an angle relative to the transverse axis of article of footwear 300. For example, LED 350 of the foremost row that is closest to lateral side 301 may be disposed rearwardly (i.e., closer to rearfoot portion 306) of LED 350 of the foremost row that is closest to medial side 303. In some embodiments, the foremost row is disposed rearwardly of motor 330 in forefoot region 302.

In some embodiments, a second row disposed adjacent to the foremost row comprises four LEDs 350, as shown in FIGS. 7B. The second row of LEDs 350 may be disposed between two straps 320. The second row of LEDs 350 may be disposed in forefoot region 302, midfoot region 304, or spanning across forefoot region 302 and midfoot region 304. In some embodiments, no motors 330 are disposed between the foremost row and the second row of LEDs 350. In some embodiments, the second row of LEDs 350 is disposed at an angle relative to the transverse axis of article of footwear 300. For example, LED 350 of the second row that is closest to lateral side 301 may be disposed rearwardly (i.e., closer to rearfoot portion 306) of LED 350 of the second row that is closest to medial side 303. In some embodiments, the second row is disposed forwardly of motor 330 in midfoot region 304.

In some embodiments, a third row disposed adjacent to the second row comprises three LEDs 350, as shown in FIGS. 7B. The third row of LEDs 350 may be disposed below or just behind a rearward strap 320. The third row of LEDs 350 may be disposed in midfoot region 304. In some embodiments, motor 330 in midfoot region is disposed between the second row and the third row of LEDs 350. In some embodiments, the third row of LEDs 350 is disposed at an angle relative to the transverse axis of article of footwear 300. For example, LED 350 of the third row that is closest to lateral side 301 may be disposed rearwardly (i.e., closer to rearfoot portion 306) of LED 350 of the third row that is closest to medial side 303. In some embodiments, the second row is disposed forwardly of motor 330 in midfoot region 304.

In some embodiments, a fourth (or rearmost) row disposed adjacent to the third row comprises three LEDs 350, as shown in FIGS. 7B. The rearmost row of LEDs 350 may be disposed in midfoot region 304, in rearfoot region 306 or spanning across midfoot region 306 and rearfoot region 306. In some embodiments, motor 330 between midfoot region 304 and rearfoot region 306 region is disposed between the third row and the rearmost row of LEDs 350. For example, motor 330 between midfoot region 304 and rearfoot region 306 may be disposed immediately forward of rearmost row of LEDs 350. In some embodiments, the rearmost row of LEDs 350 is disposed at an angle relative to the transverse axis of article of footwear 300. For example, LED 350 of the rearmost row that is closest to lateral side 301 may be disposed rearwardly (i.e., closer to rearfoot portion 306) of LED 350 of the rearmost row that is closest to medial side 303.

Other arrangements of LEDs 350 may also be used. LEDs may be positioned to provide targeted light therapy to certain portions of the wearer's foot (similar to the vibration targets discussed above).

In addition to or alternatively to the LEDs 350, in some embodiments, article of footwear 300 may incorporate one or more materials for energy sources, such as graphene, carbon fiber, ceramics, or the like, to generate far infrared radiation (FIR) through heating for similar therapeutic benefits as discussed above. In some embodiments, materials such as graphene, carbon fiber, and/or ceramics may provide additional structural support to bear the weight of the wearer of the article of footwear 300. In some embodiments, the support structure 455 may include one or more FIR-generating materials and/or heating elements for therapeutic purposes. Heat may provide a thermal-based therapeutic benefit to help with pain, joint stiffness and tension, reducing muscle spasms and muscle tone and improving blood flow. The aforementioned benefits fall under the umbrella of recovery from physical activity and the treatment of both acute and chronic conditions associated with pain and inflammation. Thermotherapy applied between the temperatures of 37 and 43 degrees Celsius can elicit the desired therapeutic effects while still being safe to users as they fall below the 44 degrees Celsius, which is the generally accepted maximum temperature before the user starts to experience adverse effects and pain.

Materials producing far infrared radiation (FIR) in article of footwear 300 may provide therapeutic benefits to the user, including but not limited to reduced chronic muscle and joint pain and fatigue, wound healing, enhanced peripheral blood flow, and muscle tissue oxygenation. FIR is a band (50 to 1000 μm) in the infrared (IR) spectrum of electromagnetic radiation; the therapeutic fluence of FIR depends on the treatment duration as well as the two possible form factors of the device. The first being an electronically operated device which elicits greater FIR power densities in the tens of mW/cm2 range by heating up a piece of graphene or ceramic material. The second form factor, utilizing textiles infused with germanium or similar materials, relies on energy produced by the body. This second form factor generates much lower power densities, being in the 0.1-5 mW/cm2 range.

In some embodiments, as shown, for example in FIG. 7C, article of footwear comprises a charging port 372. Charging port 372 is coupled to control assembly 340 and may be configured to receive a charger to charge a battery of control assembly 340. In some embodiments, charging port 372 is a USB-C port. Other types of ports may also be used for charging port 372. For example, charging port 372 may be a USB-A port, a DC jack connector, or other type of cable connector. Charging port 372 may be accessible from an exterior of article of footwear 300. For example, charging port 372 may be disposed on an exterior wall of medial side 303 of sole 310.

In some embodiments, a switch 374 is coupled to control assembly 340 and configured to turn motors 330 and LEDs 350 on and off. In some embodiments, switch 374 is disposed on an exterior wall of medial side 303 of sole 310. Similar to switch 174, switch 374 may be used to transition between operating modes in addition to turning therapeutic assemblies on and off. The operating modes may include any combination of characteristics (e.g., light therapy only, vibration therapy only, turning on all or a subset of motors 330, turning on all or a subset of LEDs 350, switching between different intensities, speeds, or patterns of vibration for motors 330, switching between different intensities of LEDs 350, a combination of the foregoing, etc.). In some embodiments, multiple switches 374 may be included. Switch 374 may have other characteristics of switch 174 described above. For example, switch 374 may be a button 374 (e.g., a button that is pushed to operate).

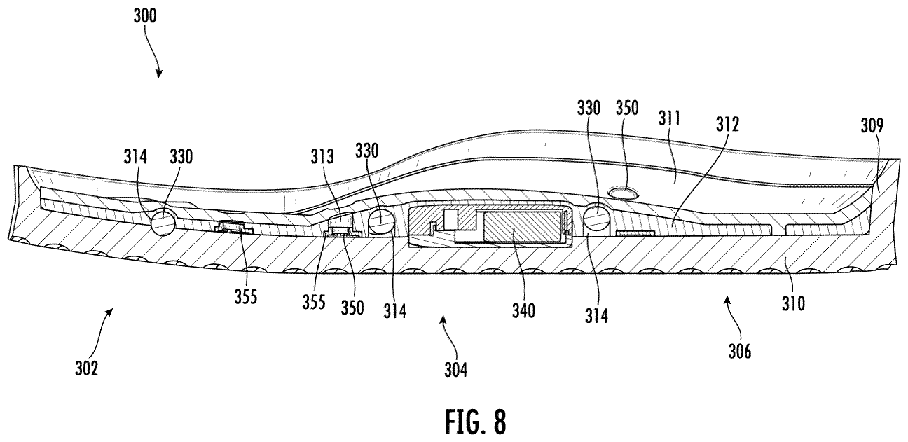

Additional details of the inner arrangement of article of footwear 300 (e.g., motors 330 and LEDs 350) are illustrated with reference to FIGS. 8-10. FIG. 8 shows a cross section of article of footwear 300 (without including straps 320). Sole 310 provides a bottom layer of article of footwear 300 (e.g., a layer configured to contact the ground). Sole 310 is disposed beneath motors 330 and LEDs 350. In some embodiments, sole 310 is concave such that it receives other components of article of footwear 300 (e.g., control assembly 340, motors 330, LEDs 350, top layer 311, etc.). For example, sole 310 may include sidewalls 309 that extend from a base of sole 310 above other components of article of footwear 300. In some embodiments, sole 310 may be made of ethyl-vinyl acetate (EVA) or other material suitable for shoe/slide soles.

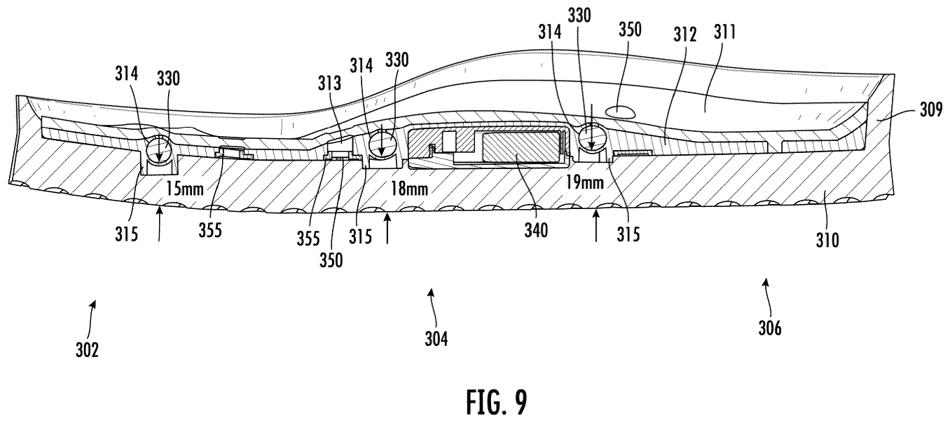

The thickness of sole 310 may be different in various embodiments. A greater thickness of sole 310 may strengthen sole 310, particularly when a wearer is standing or putting weight on article of footwear 300. In some embodiments, a greater thickness helps facilitate vibration of motors 330, even when a wearer is standing. In some embodiments. as shown, for example, in FIG. 9, a thickness of sole 110 may be provided such that a distance between a bottom of motor 330 in forefoot region 302 and a base of sole 310 is between 12 mm and 20 mm (e.g., 15 mm). In some embodiments, a thickness of sole 110 may be provided such that a distance between a bottom of motor 330 in midfoot region 304 and a base of sole 310 is between 15 mm and 22 mm (e.g., 18 mm). In some embodiments, a thickness of sole 110 may be provided such that a distance between a bottom of motor 330 (disposed between midfoot region 304 and rearfoot region 306) and a base of sole 310 is between 15 mm and 22 mm (e.g., 19 mm). Other thicknesses may also be used.

Referring again to FIG. 8, in some embodiments, article of footwear 300 comprises a mounting layer 312 disposed between top layer 311 and sole 310. In some embodiments, mounting layer 312 is made of EVA. Motors 330 may be coupled to mounting layer 312. For example, in some embodiments, mounting layer 312 includes pockets 314 for each motor 330, as shown in FIG. 8. In some embodiments, as shown in FIG. 9, pockets 314 include a spacer 315. Spacer 315 may be a protrusion of mounting layer 312 that extends below motor 330. In some embodiments, spacer 315 provides additional space for motor 330 to vibrate, even when article of footwear is being compressed (e.g., from the weight of a wearer).

Referring again to FIG. 8, in some embodiments, mounting layer 312 includes holes 313 aligned with each LED 350, allowing light from LED 350 to reach (and shine through) transparent top layer 311. In some embodiments, each LED 350 may be disposed within hole 313 of mounting layer 312. In some embodiments, article of footwear 300 comprises a support structure 355 (such as a bracket 355) for LEDs 350. Bracket 355 may be configured to protect LEDs 350. In some embodiments, each row of LEDs 350 may have its own bracket 355. In some embodiments, each LED 350 may have its own bracket 355. In some embodiments, bracket 355 is one piece shaped to protect the entire array of LEDs 350.

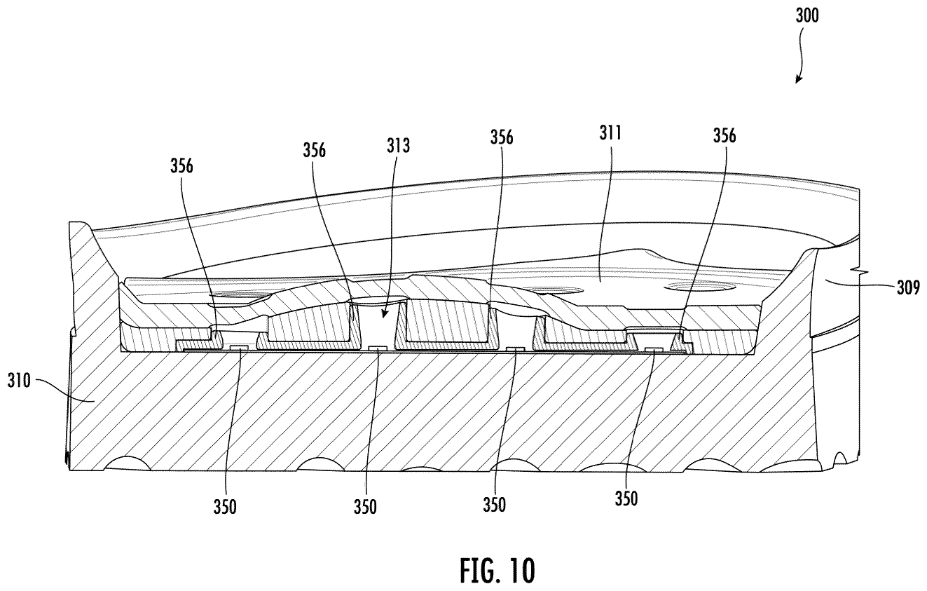

In some embodiments, bracket 355 may provide a base on which LED 350 may rest. In some embodiments, LEDs 350 rest on sole 310 and bracket 355 is disposed to surround LEDs 350 (e.g., around the sides of LEDs). In either of these situations, bracket 355 may extend around portions of LED 350, for example, to guide light from LED 350 up through hole 313 of mounting layer 312. In some embodiments, as shown, for example, in FIG. 10, bracket 355 comprises a hollow guide 356 that protrudes up through hole 313. In some embodiments, an internal portion of guide 356 may have a larger diameter towards the top of hole 313 than at a bottom of hole 313. This arrangement may provide an increased angle of light that can emanate from LED 350 through guide 356 (than if the inner diameter was constant), which may provide a higher intensity of LED light reaching a wearer, thus improving the light therapy.

Bracket 355 may be coupled to mounting layer 312, sole 310, or both (e.g., with an adhesive). In some embodiments, bracket 355 is simply placed in location (e.g., in holes 313 of mounting layer 312 and/or above sole 310) without a direct coupling mechanism. In some embodiments, bracket 355 is made of a plastic material, such as polycarbonate. In some embodiments, bracket 355 is white to reduce the amount of light from LED 350 that is absorbed. Using a white bracket 355 may help improve the light therapy by increasing the intensity of LED light reaching a wearer.

In some embodiments, control assembly 340 is disposed between mounting layer 312 and sole 310. Sole 310 and/or mounting layer 312 may include cavities or depressions to make space for control assembly 340. Control assembly 340 may be coupled to mounting layer 312, sole 310, or both. In some embodiments, control assembly 340 is simply placed in location (e.g., between mounting layer 312 sole 310) without a direct coupling mechanism.

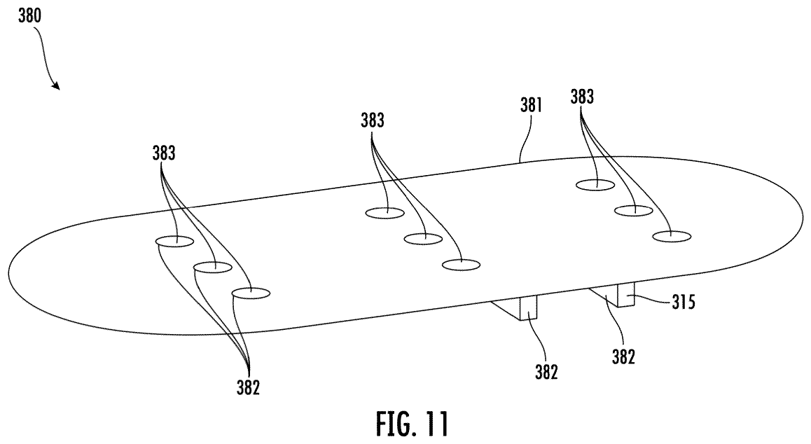

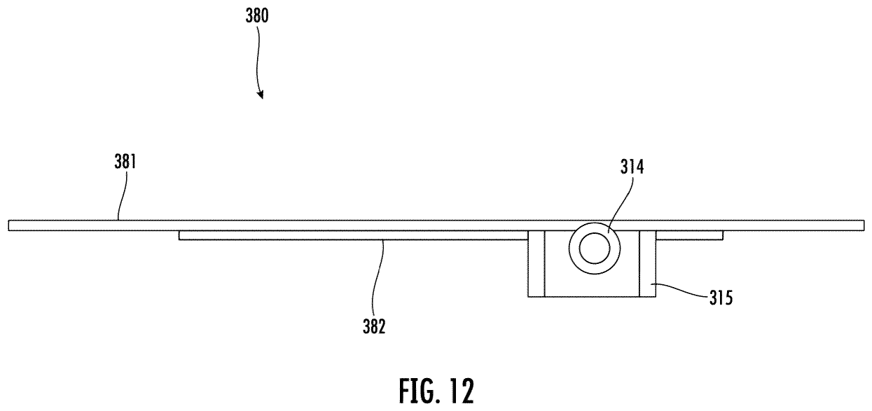

In some embodiments, rather than a transparent top layer 311 (e.g., made of silicone) and a separate mounting layer 312 (e.g., made of EVA), article of footwear 300 may include a single part made of polyurethane (e.g., thermoplastic polyurethane (TPU)) and a transparent polycarbonate. For example, as shown in FIGS. 11 and 12, a single part 380 may be made that provides a soft layer 381 as a top surface. Layer 381 may be made of a TPU material and may be soft to provide for the wearer's comfort. In some embodiments, the same material that is used for layer 381 may be used to form pockets 314 (see FIG. 12).

In some embodiments, single part 380 may be additionally made of a second material 382 that is transparent (e.g., transparent polycarbonate). Second material 382 may be disposed in locations within layer 381 as transparent covers 383 to fill holes in layer 381 that align with LEDs 350 (see FIG. 11). Thus, while layer 381 is not transparent, transparent covers 383 allow LED light to pass through to provide light therapy to a wearer's feet. In some embodiments, second material 382 may also be used to provide structure underneath layer 381 that provides structural support to protect LEDs 350. In some embodiments, second material 382 is used to form spacers 315 to create support and empty space around motors 330 to facilitate their vibration (even when a wearer's weight is on article of footwear 300) and also to reduce noise.

An article of footwear 400 according to some embodiments is shown in FIGS. 13A-13B having both a light therapy assembly and a vibration assembly. Features discussed with respect to article of footwears 100 and/or 300 above may also be implemented as part of article of footwear 400. Similarly, features discussed with respect to article of footwear 400 below may also be implemented as part of article of footwears 100 and/or 300. In addition, while article of footwear 400 comprises both a light therapy assembly and a vibration assembly, in some embodiments, an article of footwear comprises a light therapy assembly (such as the one discussed with respect to article of footwear 400) and does not include a vibration assembly.

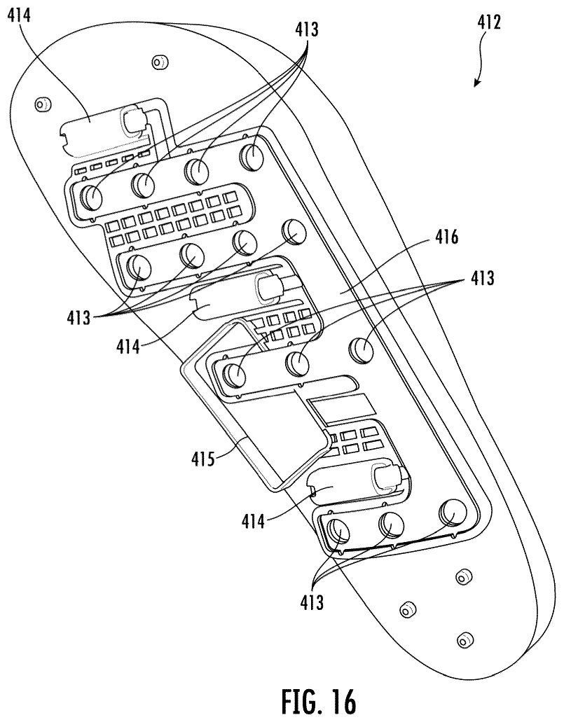

As shown in FIG. 13A, article of footwear 400 may have a sole 410 and one or more straps 420. In some embodiments, article of footwear 400 comprises a vibration assembly, which may have the features discussed above with respect to article of footwear 100 or article of footwear 300, and a light therapy assembly, which may have the features discussed above with respect to article of footwear 300. In some embodiments, article of footwear comprises a mounting layer 412. Mounting layer 412 may have some or all of the features of single part 380, and some or all of the features of mounting layer 112, or a combination of single part 380 and mounting layer 112. For example, mounting layer 412 may comprise holes 413 (see FIG. 16) and transparent cover(s) 483 (see FIG. 13A) may be disposed in holes 413 at locations that are aligned with LEDs to allow for light therapy to be applied to a wearer's foot.

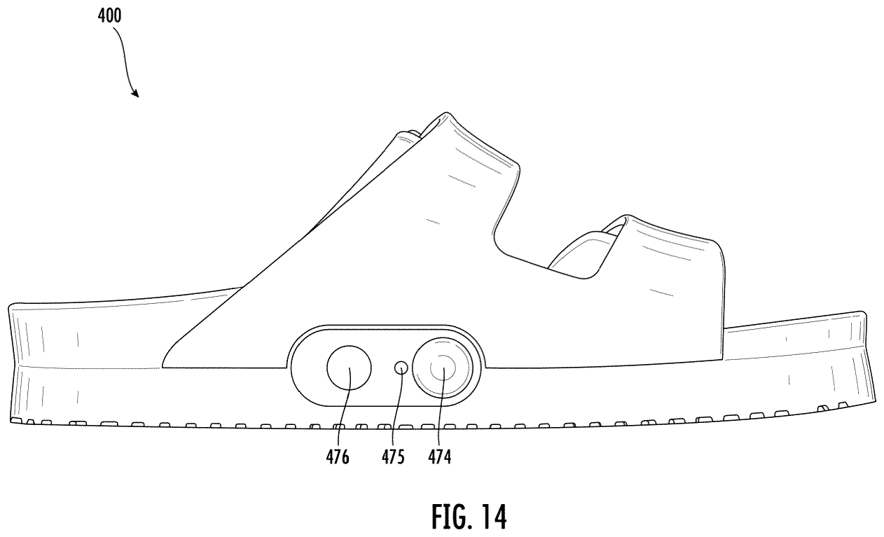

In some embodiments, as shown in FIG. 13B, a switch 474 may be disposed on an exterior wall of sole 410. Switch 474 may have the characteristics of switch 174 or 374. Unlike article of footwear 300, article of footwear 400 may use wireless charging (as discussed below) instead of having a charging port. In some embodiments, article of footwear 400 comprises a status light 475 disposed adjacent to switch 474. Status light 475 may provide information to a user (e.g., low battery, fully charged, turned on, modes of operation, etc.) based on a color and/or a pattern of status light 475. In some embodiments, as shown, for example, in FIG. 14, article of footwear 400 may include two switches, a switch 474 for the vibration assembly and a switch 476 for the light therapy assembly. Thus, switch 474 may be used to turn vibration motors on and off (and switch between various modes of operation), and switch 476 may be used to turn LEDs on and off (and switch between various modes of operation).

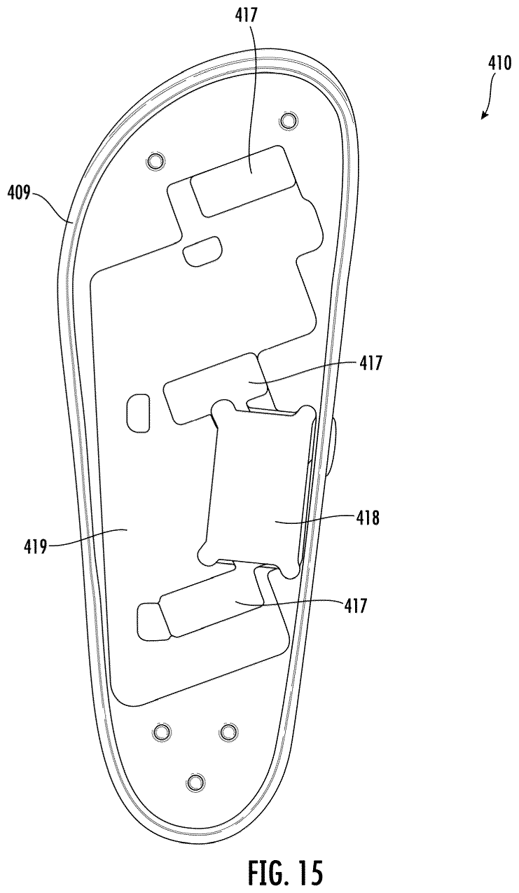

Additional details of sole 410 are shown, for example, in FIG. 15. In some embodiments, sole 410 comprises a sidewall 409. Sidewall 409 may be a raised edge around sole 410. Sidewall 409 may extend above other components of article of footwear 400 (e.g., mounting layer 412). In some embodiments, a top surface of sole 410 may define one or more cavities configured to receive other portions of article of footwear 400. For example, top surface of sole 410 may define a cavity 417 for each motor in the vibration assembly of article of footwear 400. In FIG. 15, sole 410 defines three cavities 417 for motors. In some embodiments, top surface of sole 410 defines a cavity 418 configured to receive all or a portion of a control assembly for the vibration assembly and light therapy assembly of article of footwear 400. In some embodiments, top surface of sole 410 defines a cavity 419 for the LEDs (and associated structure) of the light therapy assembly of article of footwear 400. In some embodiments, cavities 417, 418, and 419 may overlap or be nested within each other. For example, cavity 418 may be the deepest cavity and may partially overlap with one or more cavities 417 and with cavity 419. Cavities 417 may be deeper than cavity 419 and may partially or entirely overlap with cavity 419. In some embodiments, a portion of cavities 417 is configured to provide extra space to allow motors to vibrate, even when article of footwear 400 is being compressed.

Additional details of mounting layer 412 are shown, for example, in FIG. 16. In some embodiments, mounting layer 412 defines holes 413 therethrough. Holes 413 may be positioned to align with LEDs of the light therapy assembly of article of footwear 400. In some embodiments, mounting layer 412 comprises pockets 414 configured to hold motors of the vibration assembly of article of footwear 400. Pockets 414 may be disposed on a bottom of mounting layer 412. In FIG. 16, mounting layer 412 comprises three pockets 414 for motors. In some embodiments, a bottom surface of mounting layer 412 defines a cavity 415 configured to receive all or a portion of a control assembly for the vibration assembly and light therapy assembly of article of footwear 400. In some embodiments, a bottom surface of mounting layer 412 defines a cavity 416 for the LEDs (and associated structure) of the light therapy assembly of article of footwear 400.

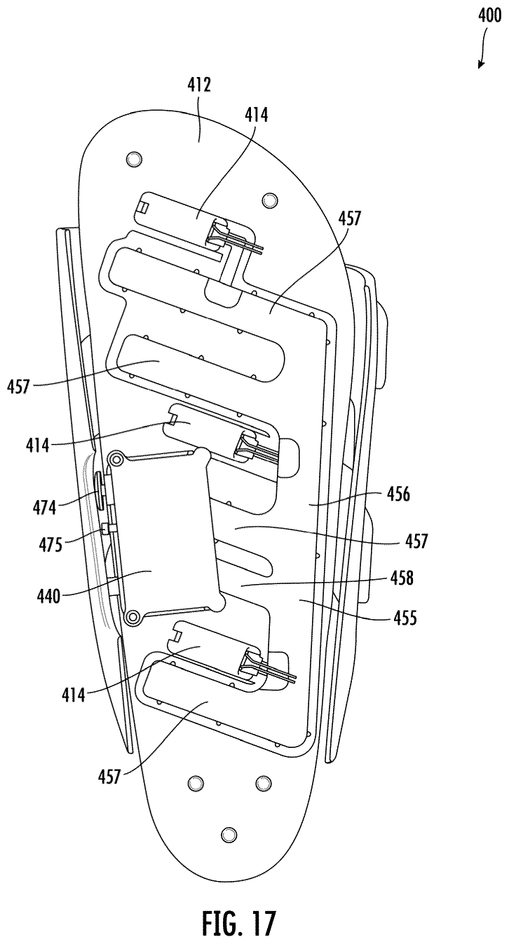

Additional details relating to the light therapy assembly is shown, for example, in FIGS. 17-19. FIG. 17 shows a bottom view of article of footwear 400 with sole 410 removed. Portions of mounting layer 412 are visible, including pockets 414 with cabling of the motors extending out of the pockets 414. Other portions of mounting layer 412 are covered by a control assembly 440 and support structure 455. In some embodiments, control assembly 440 is disposed to be aligned with cavity 415 (see FIG. 16). Control assembly 440 may be operably connected with switch 474 and status light 475.

In some embodiments, support structure 455 comprises structure (e.g., bracket 455) to contain LEDs. In some embodiments, bracket 455 comprises a longitudinal section 456 that extends longitudinally along a side of article of footwear 400. For example, longitudinal section 456 may extend from a rearfoot portion of article of footwear 400 to a forefoot portion of article of footwear 400. Branching from longitudinal section 456 is a series of transverse sections 457 extending transversely across article of footwear 400. Each transverse section 457 may be associated with a row of LEDs. Thus, the number of transverse sections 457 may be equal to the number of rows of LEDs in article of footwear 400. In some embodiments, a control assembly section 458 also extends transversely from longitudinal section 456 to control assembly 440. An electrical connection from control assembly 440 to LEDs may be contained within bracket 455. In some embodiments, an electrical connection from control assembly 440 to motors of article of footwear 400 may also be contained within bracket 455. For example, the cabling extending from pockets 414 may extend into bracket 455. In some embodiments, bracket 455 is U-shaped to be disposed below and to the sides of LEDs when article of footwear 400 is worn.

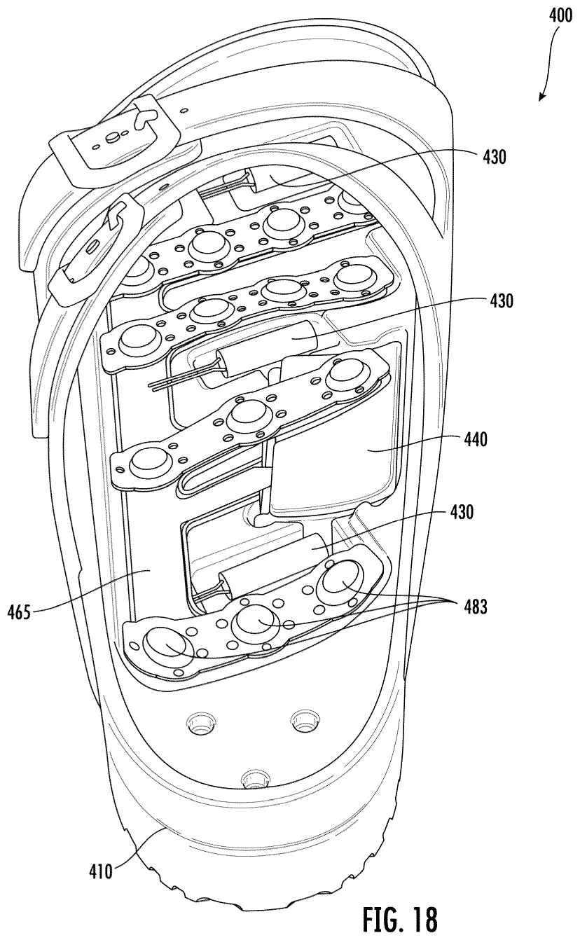

FIG. 18 shows a top view of article of footwear 400 with mounting layer 412 removed. In some embodiments, transparent covers 483 (which are configured to fill holes 413 in mounting layer 412) are disposed over bracket 455. In some embodiments, transparent cover 483 is configured to mate with bracket 455 so as to enclose LEDs within bracket 455 and transparent cover 483. In some embodiments, a row of transparent covers 483 may be one piece, as shown in FIG. 18. Each row of transparent covers 483 may correspond to a row of LEDs, a row of holes 413, and a transverse section 457 of bracket 455. In some embodiments, transparent covers 483 comprise lenses for LEDs.

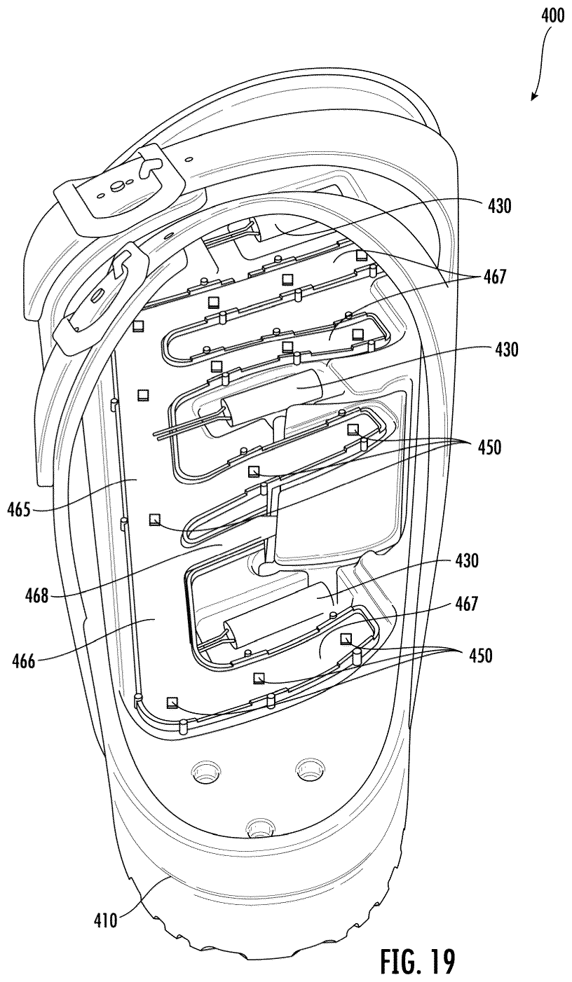

FIG. 19 shows a similar view as FIG. 18, but transparent covers 483 are also removed to reveal LEDs 450 and LED support 465. LED support 465 may provide a physical and electrical connection between each LED 450 and control assembly 440. In some embodiments, LED support 465 is shaped to fit within bracket 455. LEDs 450 may rest on top of LED support 465. In some embodiments, LED support 465 comprises a longitudinal section 466 that extends longitudinally along a side of article of footwear 400. For example, longitudinal section 466 may extend from a rearfoot portion of article of footwear 400 to a forefoot portion of article of footwear 400. Branching from longitudinal section 466 is a series of transverse sections 467 extending transversely across article of footwear 400. Each transverse section 467 may be associated with a row of LEDs 450. Thus, the number of transverse sections 467 may be equal to the number of rows of LEDs 450 in article of footwear 400. In some embodiments, portions of longitudinal section 466 disposed between transverse sections 467 may be softer than other portions of LED support 465. This may help make electrical connections between LEDs 450 more reliable, particularly while a wearer is walking in article of footwear 400, and thus improve reliability of LEDs 450 themselves in providing light therapy.

In some embodiments, a control assembly section 468 also extends transversely from longitudinal section 466 to control assembly 440. LED support 465 may provide an electrical connection from control assembly 440 to LEDs 450. In some embodiments, LED support 465 may also provide a portion of an electrical connection from control assembly 440 to motors 430 of article of footwear 400. For example, cabling of motors 430 may electrically connect to LED support 465.

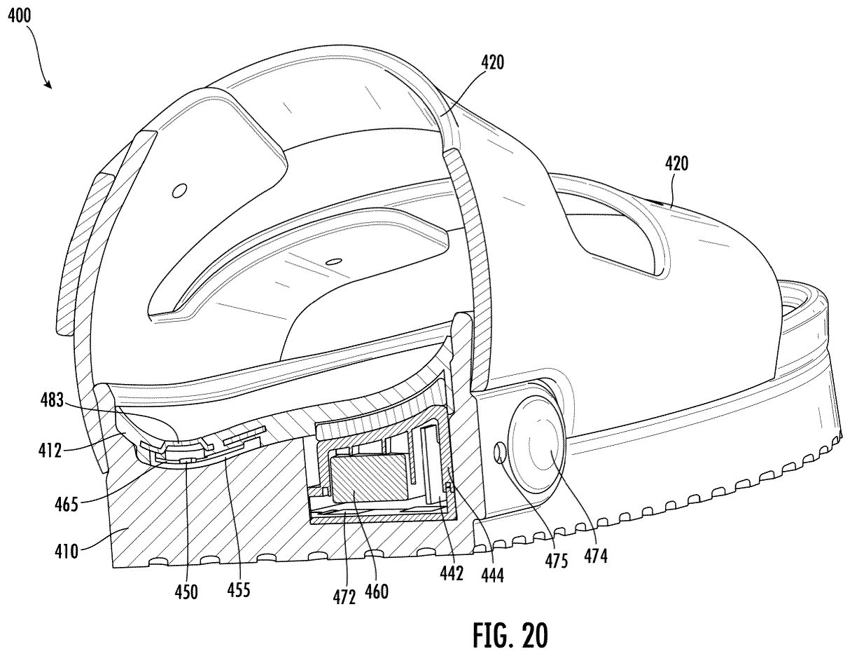

The cross-section view shown in FIG. 20 shows various components of the light therapy assembly of article of footwear 400 assembled together (including transparent cover 483, mounting layer 412, LED 450, LED support 465, bracket 455, sole 410, and control assembly 440). Control assembly 440 may include a protective housing 444, control circuitry 442 (e.g., a printed circuit board (PCB)), and a battery 460 (which may have the features discussed above for protective housing 150, battery 160, and control circuitry 170, while also powering and controlling operation of light therapy assembly). In some embodiments, control circuitry 442 may include a wireless communication module (e.g., for Bluetooth connection, near-field communication (NFC), Wi-FI connection, or the like). In some embodiments, each shoe in a pair of articles of footwear 400 may connect or pair with each other and work in tandem via a connection through the wireless communication module in control circuitry 442. In some embodiments, the wireless communication module may allow for the article of footwear 400 to connect to and communicate with an external device (e.g., a mobile device, tablet, computing device, or the like), via Bluetooth, NFC, Wi-Fi, or other networks. In some embodiments, a wearer of the article of footwear 400 may control operation of the article of footwear 400 (e.g., powering on/off the footwear, powering on/off the vibration and/or light therapies, adjusting the levels of vibration and/or light therapies, etc.) through the external device that is connected to the article of footwear 400. In some embodiments, the wearer of the article of footwear 400 may program or select a schedule of treatment for the vibration and/or light therapies in the footwear 400 using the interface of the connected external device. In some embodiments, the schedule of treatment may include a combination of time intervals and/or varying durations for treatment, such as for example, vibration and/or light therapy for a duration of 10 minutes every hour for 5 hours.

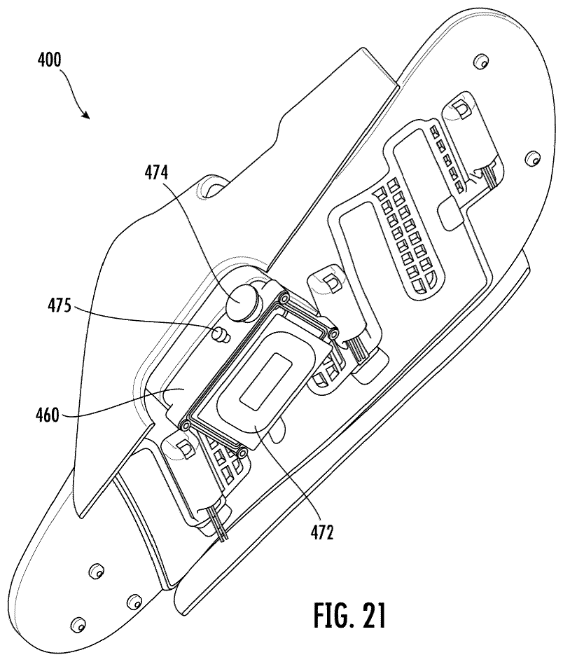

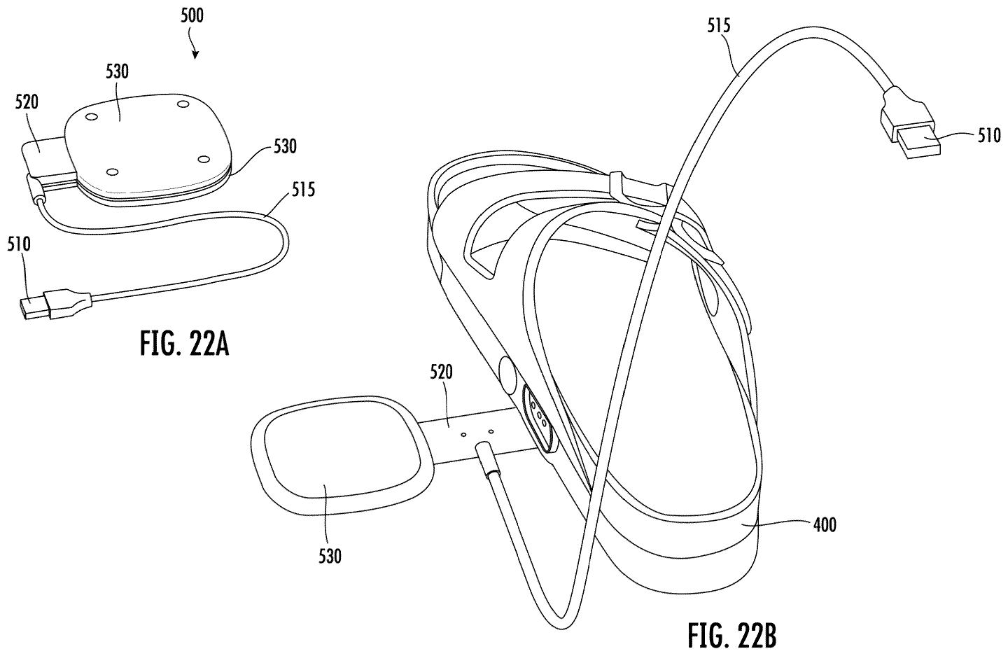

In some embodiments, a wireless charging module 472 (as shown in FIGS. 20 and 21) is disposed underneath battery 460. Wireless charging module 472 is configured to charge battery 460 when paired with a charging device 500 (see FIGS. 22A and 22B). In some embodiments, charging device 500 comprises a connector 510, a cable 515, a flexible portion 520, and one or more charging pads 530. In some embodiments, connector 510 may be configured to plug into a power source (e.g., a power outlet, a USB port, etc.). In some embodiments, connector 510 comprises a USB connector. In some embodiments, cable 515 extends from connector 510 to flexible portion 520. Flexible portion 520 may be disposed between two charging pads 530, allowing for charging device 500 to fold into a more compact configuration (see FIG. 22A). Having two charging pads 530 allows for a pair of articles of footwear 400 to be charged simultaneously. Each article of footwear 400 is placed on a charging pad 530 such that charging pad 530 is aligned with charging module 472 is aligned with charging pad 530 (see FIG. 22B). Thus, article of footwear 400 may be charged wirelessly (instead of with a charging port). In some embodiments, wireless charging of article footwear 400 may allow for the device's electronics to be sealed and waterproof.

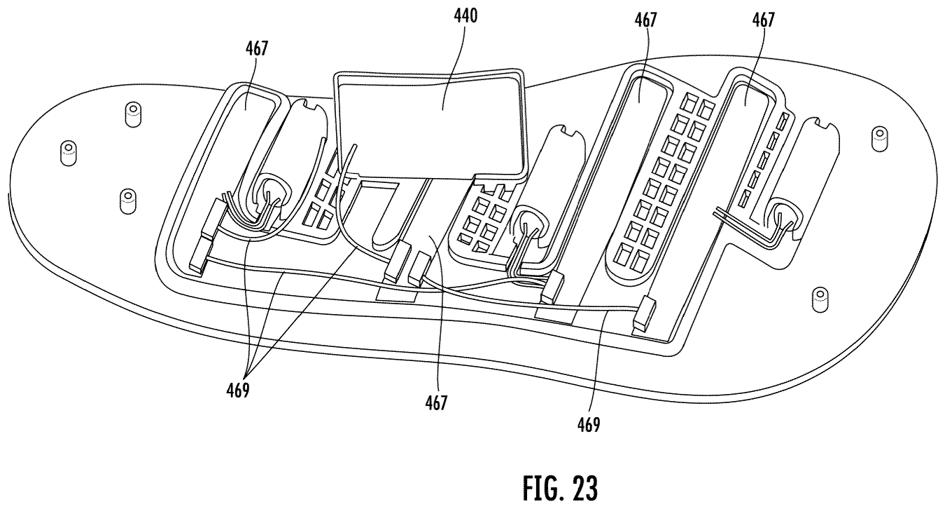

Variations to article of footwear 400 are also possible. For example, instead of having a single LED support 465 with a longitudinal section 466, each transverse section 467 may be its own LED support, as shown, for example, in FIG. 23. In place of longitudinal section 466 and control assembly section 468, an electrical connection from control assembly 440 may be provided with cables 469 to connect each transverse section 467. This configuration may also make the electrical connections between LEDs 450 more reliable, particularly while a wearer is walking in article of footwear 400, and thus improve reliability of LEDs 450 themselves in providing light therapy.

Other types of therapy may be provided with an article of footwear in some embodiments, either in addition to or instead of vibration and/or light therapy. As discussed above, in some embodiments, an article of footwear may include material(s) to generate far infrared radiation (FIR) for heating. In some embodiments, an article of footwear may provide both heating (e.g., via FIR, heat spreaders, or use of thermoelectric cooler(s) (TEC)) and cooling (e.g., via TEC(s)) for therapeutic effect.

FIR heating uses far infrared radiation to generate heat, which is a form of electromagnetic radiation with wavelengths longer than visible light but shorter than microwaves. The benefits of FIR heating include improved blood circulation, relief from muscle and joint pain, detoxification, and relaxation. Unlike conventional heating that warms the air around a person, FIR heating directly warms the body, leading to more efficient heat transfer and a more comfortable experience. FIR technology utilizes specialized materials or devices to emit this radiation safely and effectively.

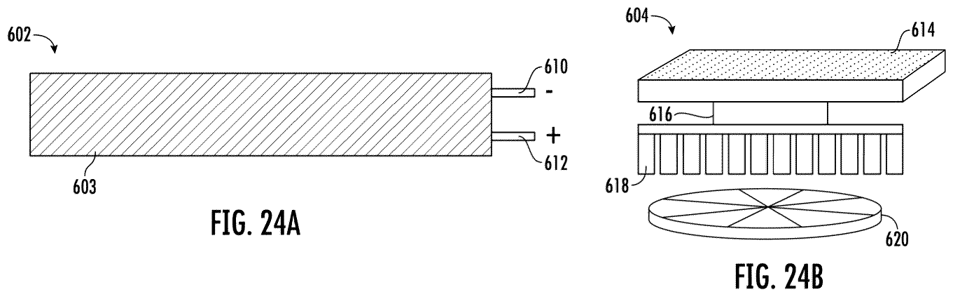

FIG. 24A shows a schematic view of a far infrared (FIR) heating system 602 according to some embodiments. FIR heating system 602 has body 603 and terminals 610 and 612. Body 603 may be formed of any number of materials, including carbon fiber, graphene, or ceramic. In some embodiments, such as the embodiment shown in FIG. 24A, body 603 may be formed of a mesh. The components or strands of the mesh may be formed of carbon fiber, graphene, or ceramic and may be arranged in a way such that the mesh carries a current. In some embodiments, the mesh of body 603 may be formed by carbon fiber strands arranged in various patterns, including sinusoidal or wave patterns arranged in horizontal and/or vertical configurations. In some embodiments, FIR heating system 602 generates heat by passing a current through body 603. Terminals 610 and 612 connect body 603 to a current source.

A Thermoelectric Cooler (TEC) works based on the Peltier effect, which occurs when an electric current passes through two different conductive materials, creating a temperature difference. This temperature difference allows one side of the TEC to absorb heat (cooling it down) while the other side dissipates heat (increasing its temperature).

A TEC typically consists of multiple thermoelectric junctions sandwiched between two ceramic plates. When a voltage is applied, electrons move from the cooler side (cold side) to the heat sink side (hot side), transferring heat away from the area to cool. This makes TECs suitable for applications like electronic cooling, portable refrigerators, and temperature control in various devices. The efficiency of a TEC is influenced by the temperature difference between the two sides and the materials used in its construction. In addition, changing the direction of the current will change which side of the TEC is the cold side and the hot side. Thus, TECs may be used for both cooling and for heating.

FIG. 24B shows a schematic view of thermoelectric cooler assembly 604 according to some embodiments. Thermoelectric cooler assembly 604 includes contact area 614, thermoelectric cooler 616, heat sink 618, and fan 620. In some embodiments, contact area 614 is configured to contact the foot of the wearer to facilitate heat transfer from the wearer's foot to thermoelectric cooler 616 (or from thermoelectric cooler 616 to the wearer's foot). As used here, contact area 614 may be in “contact” with the foot of the wearer even if a layer of the article of footwear (such as, for example, a cloth layer), a sock, or other layers are between contact area 614 and the wearer's foot. Thermoelectric cooler assembly 604 also includes thermoelectric cooler 616. Thermoelectric cooler 616 has a hot side coupled to heat sink 618 and a cold side coupled to contact area 614. Heat sink 618 is configured to dissipate heat from the hot side of thermoelectric cooler 616. A fan 620 may move air over heat sink 618 to help remove heat from heat sink 618 into the environment.

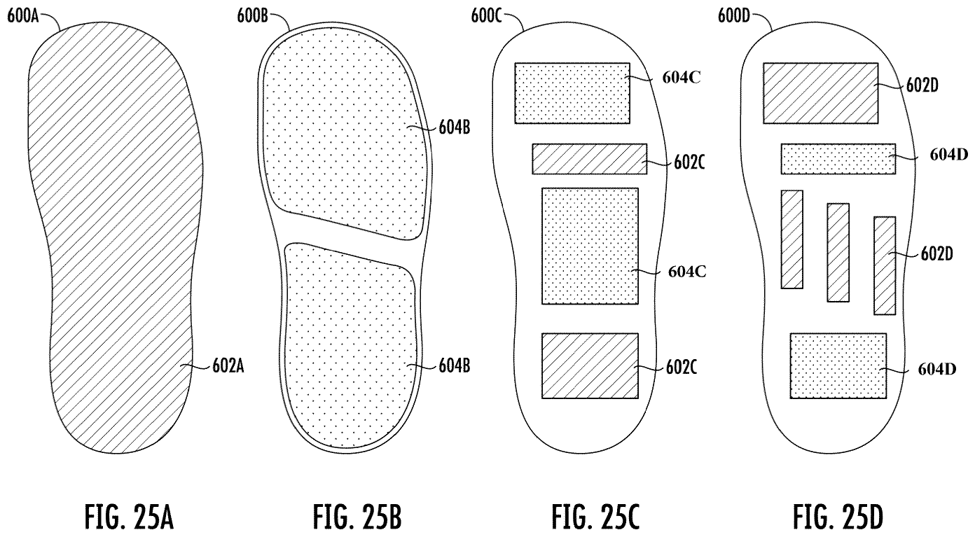

An article of footwear may include one or more FIR heating systems 602, one or more thermoelectric cooler assemblies 604, or both. FIGS. 25A-25D, for example, show schematics of embodiments of an article of footwear according to some embodiments. FIGS. 25A - 25D show areas of the sole of an article of footwear that incorporate FIR heating system 602 or thermoelectric cooler assemblies 604 according to some embodiments. FIG. 25A shows sole embodiment 600A that has FIR heating system 602A. FIR heating system 602A extends across the entire sole embodiment 600A. In this embodiment, the entire bottom of the foot of the user may be treated using FIR heating system 602A. FIGS. 25B-25C show alternative embodiments 600B-D with FIR heating systems 602C and 602D and thermoelectric cooler assemblies 604B-D.

FIG. 25B shows sole embodiment 600B. Sole embodiment 600B includes thermoelectric cooler assemblies 604B. As shown in FIG. 25B, two thermoelectric cooler assemblies 604B are disposed in sole embodiment 600B. One of thermoelectric cooler assemblies 604B is located in the forefoot portion of sole embodiment 600B and one is located in the rear foot portion. The areas of thermoelectric cooler assemblies 604B shown in FIG. 25B are the areas of contact areas 614 of thermoelectric cooler assemblies 604B. Other components of thermoelectric cooler assemblies 604B may be disposed beneath contact area 614 in such a way as to add or remove heat from contact area 614. In the embodiment shown in FIG. 25B, each thermoelectric cooler assemblies 604B takes ups roughly the same area of sole embodiment 600B. However, other embodiments are contemplated. For example, thermoelectric cooler assemblies 604B may be larger in a rear foot portion than the forefoot portion or the opposite. In some embodiments, more than two thermoelectric cooler assemblies 604B may be included.

FIG. 25C shows sole embodiment 600C. Sole embodiment 600C includes areas with FIR heating systems 602C and thermoelectrical cooler assemblies 604C. As shown in FIG. 25C, sole embodiment 600C includes two thermoelectric cooler assemblies 604C: one located in the forefoot region and one located in the midfoot region of sole embodiment 600C. Sole embodiment 600C also includes two FIR heating systems 602C: one located between the two thermoelectric cooler assemblies 604C and one located in the rear foot portion sole embodiment 600C. Although the FIR heating systems 602C and thermoelectric cooler assemblies 604C shown in FIG. 25C have square contact areas with the foot of the wearer, this is merely an example and other shapes are contemplated.

FIG. 25D shows sole embodiment 600D. Sole embodiment 600D has FIR heating systems 602D and thermoelectric cooler assemblies 604D. FIR heating systems 602D are disposed at four places in sole embodiment 600D: a first FIR heating systems 602D is disposed in the forefoot portion of sole embodiment 600D and three, parallel FIR heating systems 602D are disposed at the midfoot region of sole embodiment 600D. Two thermoelectric cooler assemblies 604D are disposed in 600D: one between the two sets of FIR heating systems 602D and one at the rear foot portion of sole embodiment 600D. In some embodiments, the thermoelectric cooler assemblies 604D and FIR heating systems 602D may be arranged in different areas to target specific areas of the wearer's foot (e.g., heel, arch, toes, etc.).

In some embodiments, a single current may power a plurality of FIR heating systems disposed in a sole. In some embodiments, a single thermoelectric cooler assembly may provide or remove heat from multiple contact areas.

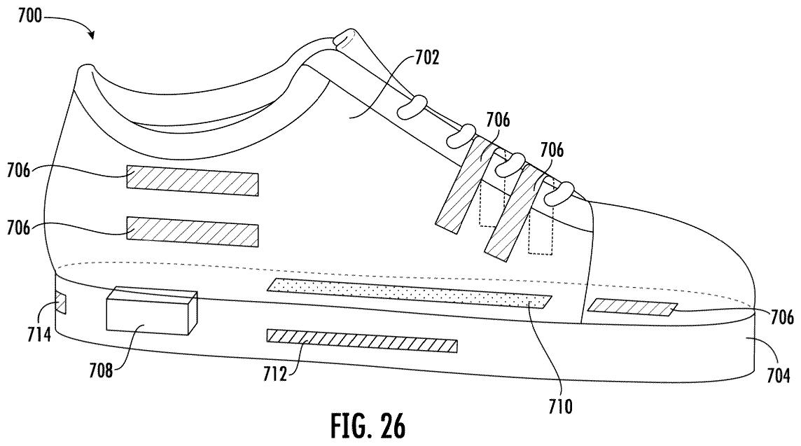

FIG. 26 shows footwear 700 according to some embodiments. Footwear 700 includes an upper 702 and a sole 704. Upper 702 and sole 704 include integrated FIR heating systems 706. In some embodiments, sole 704 may include an integrated FIR heating system 706 in a forefoot region of sole 704. In some embodiments, one or more integrated FIR heating systems 706 extend across upper 702 from a medial side to a lateral side of upper 702. In some embodiments, integrated FIR heating systems 706 may extend in a discontinuous manner from the medial side to the lateral side of upper 702. For example, integrated FIR heating systems 706 may extend across a portion of the medial side of upper 702, stop when reaching a lacing portion of footwear 700, and continue on the lateral side of upper 702. In some embodiments, each of the medial side and lateral side of upper 702 may have three or more integrated FIR heating systems 706. In some embodiments, integrated FIR heating systems 706 may be targeted to provide heat to a specific region of the foot of the wearer. For example, FIG. 26 shows integrated FIR heating systems 706 located near the ankle portion of footwear 700. These integrated FIR heating systems 706 may be used to provide a therapeutic regimen to the ankle of the wearer. Other configurations are also possible.

A thermoelectric cooling system 710 is also integrated into sole 704. Integrated FIR heating systems 706 and thermoelectric cooling system 710 are electrically and operatively coupled to controller 708. Controller 708 may include a battery configured to power integrated FIR heating systems 706 and a PCB board. Controller 708 is configured to control integrated FIR heating systems 706 and thermoelectric cooling system 710. In some embodiments, particularly embodiments with thermoelectric cooling system 710, footwear 700 may also include air intake 712. Air intake 712 is configured to draw in air, which flows across a portion of thermoelectric cooling system 710 to remove heat from thermoelectric cooling system 710. The heated air may exit from exhaust port 714. In some embodiments, footwear 700 may include more than one air intake 712. For example, footwear 700 may have several air intakes 712 formed in sole 704. In some embodiments, footwear 700 includes multiple exhaust ports 714. Exhaust port 714 may be in the rear of footwear 700 (e.g., a rear of sole 704), on a side of footwear 700 (e.g., a side of sole 704), or on the bottom of sole 704. The air may pass from air intake 712, across thermoelectric cooling system 710, and to exhaust port 714 through channels formed in sole 704.

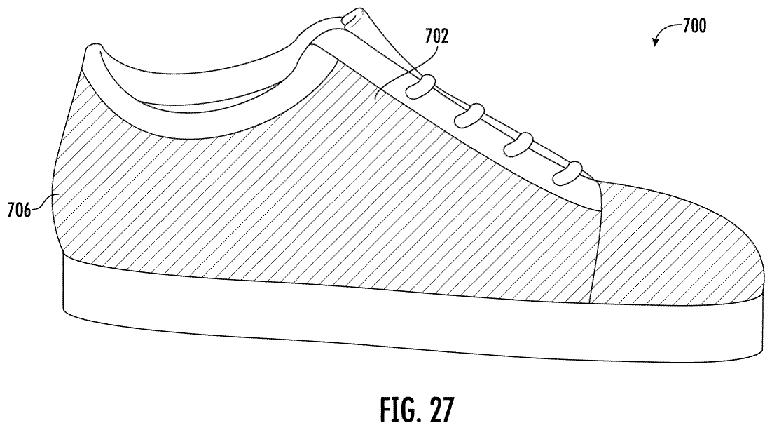

FIG. 27 shows an embodiment of footwear 700. Footwear 700 includes upper 702. Upper 702 has integrated FIR heating systems 706. In this embodiment, integrated FIR heating systems 706 comprise most of upper 702. In some embodiments, the entire upper 702 may have FIR heating elements embedded therein. In some embodiments, FIR elements may be embedded in an entire article of footwear, including the upper and the sole.

As shown by the examples above, articles of footwear may provide heat and/or cold therapy. In embodiments that include both heat and cold therapy, hot and cold may be applied sequentially rather than simultaneously. For example, in some embodiments, the article of footwear may be configured to not apply heat therapy and cold therapy simultaneously. In some embodiments, the article of footwear may be configured to provide contrast therapy, alternating between heat therapy and cold therapy.

Another type of therapy that may be provided in an article of footwear is electrical stimulation. Neuromuscular electrical stimulation (NMES) is a therapy that uses electrical impulses to cause muscle contractions. This technique involves placing electrodes on the skin over specific muscles to stimulate them. NMES offers several medical benefits: it helps strengthen weak muscles, improves muscle tone, and provides pain relief by interrupting pain signals and promoting the release of endorphins. Additionally, it increases blood circulation to the targeted area, promoting healing and reducing swelling. NMES is particularly useful for preventing muscle atrophy in individuals with limited mobility and is commonly used in rehabilitation after surgery or injury. It also aids in retraining the nervous system for better coordination and control of movements.

Transcutaneous Electrical Nerve Stimulation (TENS) is a non-invasive method used to relieve pain. It involves using low-voltage electrical currents delivered through electrodes placed on the skin's surface. Electrical impulses can help to reduce pain by stimulating the release of endorphins, the body's natural painkillers, and by interrupting pain signals sent to the brain.

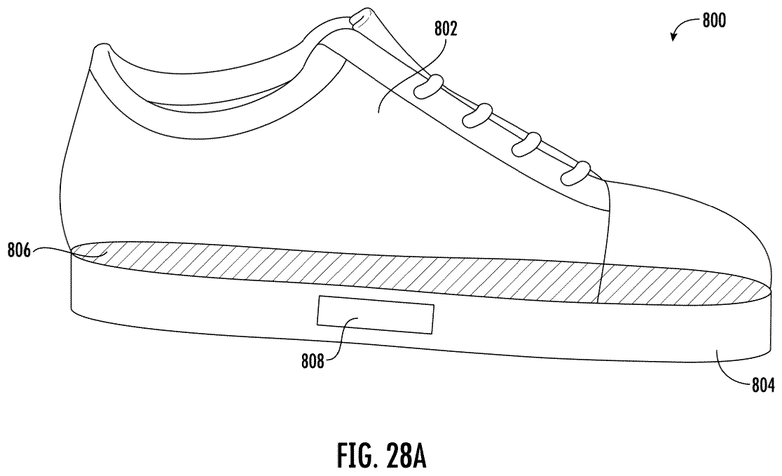

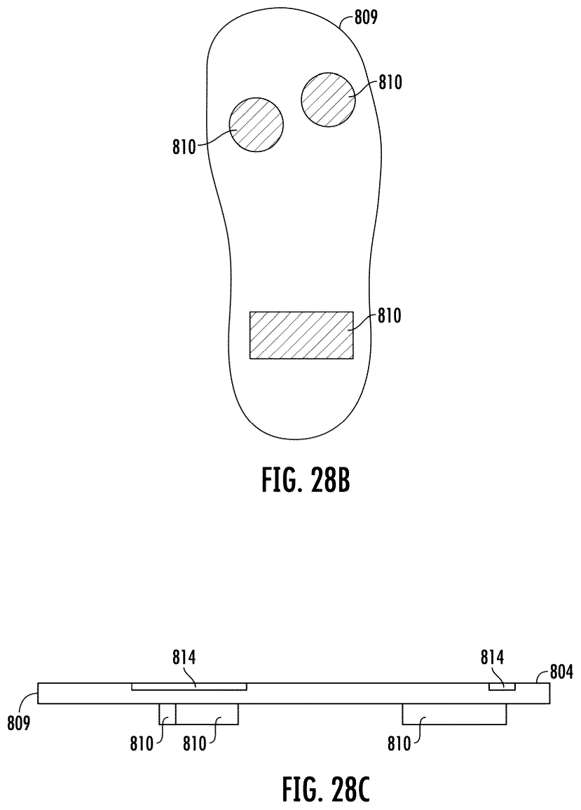

FIG. 28A shows an embodiment of footwear 800. Footwear 800 includes upper 802 and sole 804. Sole 804 includes void 806 and module 808. Module 808 may include controllers, batteries, PCB modules, or other accessories. Void 806 may be configured to receive insert 809. A bottom view of insert 809 is shown in FIG. 28B. Insert 809 is removable from sole 804 and is configured to engage void 806 to form a top portion of sole 804. In some embodiments, insert 809 may be integrated into sole 804 and non-removable from sole 804. As shown in FIGS. 28B-28C, connectors 810 extend from insert 809. Connectors 810 are configured to mate with ports in sole 804. Connectors 810 both locate insert 809 in sole 804 and are configured to permit the passage of electrical signals from sole 804 to neuromuscular electrical stimulators 814 disposed in insert 809. Neuromuscular electrical stimulators 814 are configured to provide a pulsed electromagnetic field and configured to provide a therapeutic regimen to the foot of the wearer. The therapeutic regimen may include neuromuscular electrical stimulation or transcutaneous electrical nerve stimulation. Either may be targeted to specific muscle areas or for localized pain relief. FIGS. 28A-C provide example locations for targeting electrical stimulation, but other locations may also be used.

Pulsed electromagnetic field therapy (PEMF) is a non-invasive treatment that uses low-frequency electromagnetic fields to promote healing and reduce pain. The therapy involves the application of pulsed electromagnetic fields to the body, which can stimulate cellular repair, enhance circulation, and improve tissue oxygenation. It is often used for various conditions, including chronic pain, inflammation, arthritis, and to support recovery from injuries. PEMF has also been shown to reduce pain and stimulate bone marrow growth.

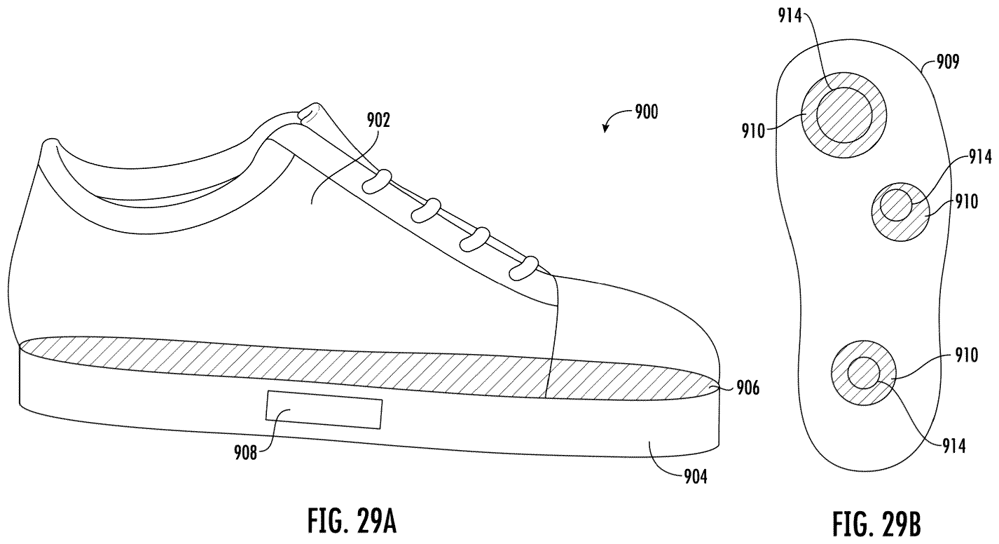

FIG. 29A shows an embodiment of footwear 900. Footwear 900 includes upper 902 and sole 904. Sole 904 includes void 906 and module 908. Module 908 may include controllers, batteries, PCB modules, or other accessories. Void 906 may be configured to receive an insert 909. A bottom view of insert 909 is shown in FIG. 28B. Insert 909 is removable from sole 904 and is configured to engage void 906 to form a top portion of sole 904. In some embodiments, insert 909 may be integrated into sole 904 and non-removable from sole 904. As shown in FIG. 29B, connectors 910 extend from insert 909. Connectors 910 are configured to mate with ports in sole 904. Connectors 910 both locate insert 909 in sole 904 and are configured to permit the passage of electrical signals from sole 904 to embedded coils 914 disposed in insert 909. Embedded coils 914 are configured to receive an electrical signal and generate a pulsed magnetic field. The pulsed magnetic field may be used to provide a PEMF therapy to a foot, or portion of a foot, of a wearer. In some embodiments, embedded coils 914 are disposed in connectors 910, as shown in FIG. 29B. In some embodiments, embedded coils 914 may be disposed in other portions of insert 909. In some embodiments, embedded coils 914 may be disposed in sole 904.

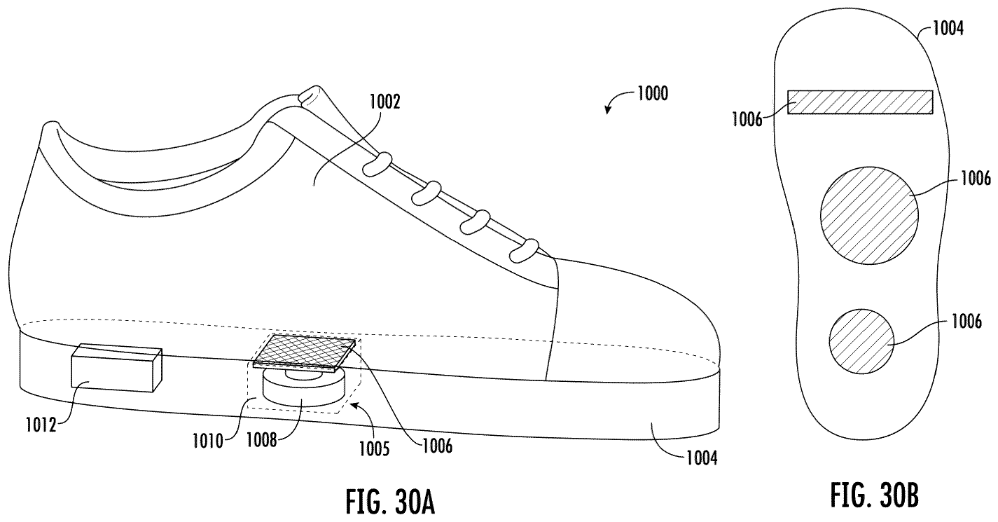

In some embodiments, articles of footwear may provide vibroacoustic treatment. FIG. 30A shows an embodiment of footwear 1000. Footwear 1000 has upper 1002 and sole 1004. Sole 1004 includes module 1012. Module 1012 may include controllers, batteries, PCB modules, or other accessories. In some embodiments, footwear 1000 includes transducer 1005. Transducer 1005 is configured to generate vibrations corresponding to specific musical/tonal frequencies. Transducer 1005 may be an assembly like the ones disclosed in U.S. Pat. Nos. 8,617,089 and 8,761,417 (the entirety of each is hereby incorporated into this application by reference thereto). Transducer 1005 may include sound heart 1008, which is configured to generate a sound and/or vibration. Transducer 1005 may be disposed in space 1010 formed in sole 1004. Space 1010 is configured to provide a resonance space for transducer 1005, allowing sound heart 1008 to vibrate. Transducer 1005 also includes resonating plate 1006. Resonating plate 1006 is configured to transmit the resonance and vibration of transducer 1005 to the foot of the wearer. Transducer 1005 may be configured to generate vibrations corresponding to specific tonal frequencies. In some embodiments, transducer 1005 may be used together with vibrating motors, such as those discussed above, or it may be used without vibrating motors. Although FIG. 30A shows one transducer 1005, other numbers of transducer 1005 may be used. For example, FIG. 30B shows a schematic view of sole 1004 with three transducers of different sizes and shapes. Sole 1004 shows transducer 1005 with resonating plate 1006 disposed in sole 1004. Transducer 1005 may be located in any number of locations in the sole 1004 (e.g., in the rearfoot, midfoot, or forefoot). For example, three transducers 1005 with resonating plates 1006 may be disposed in sole 1004 as shown in FIG. 30B.

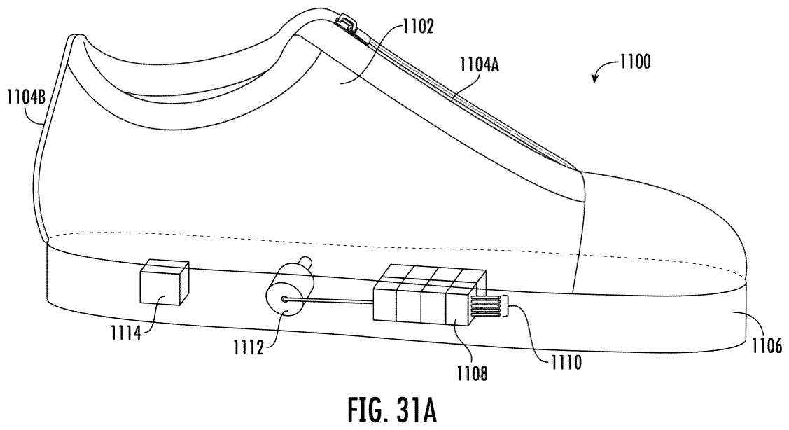

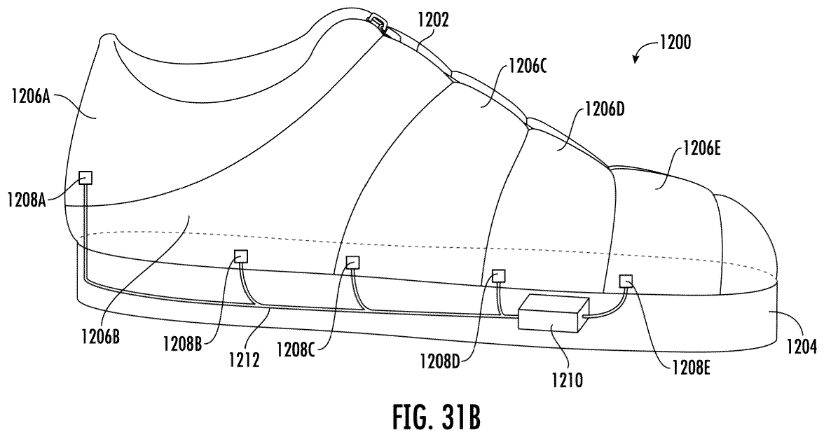

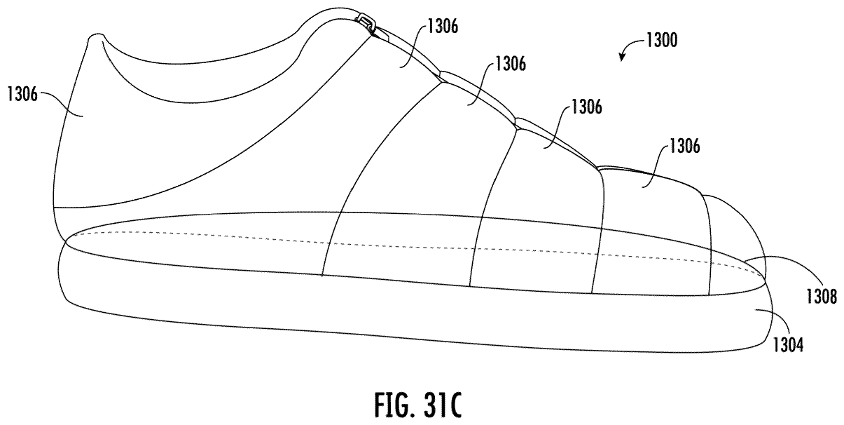

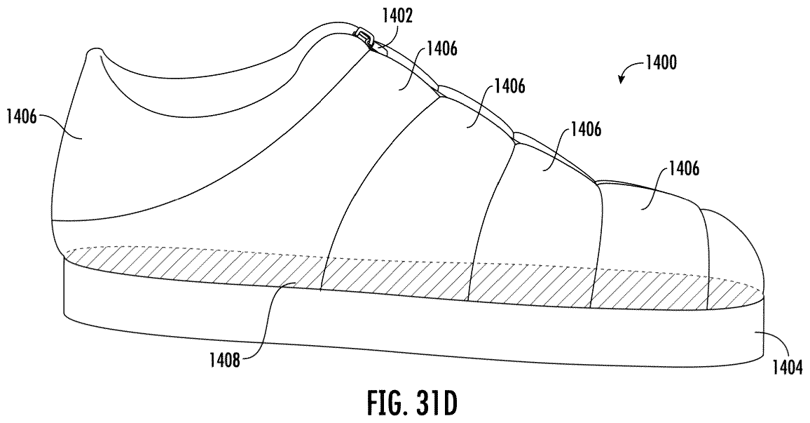

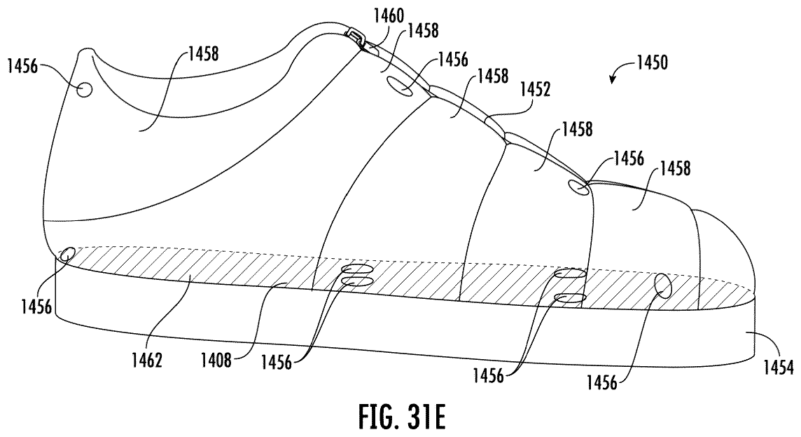

In some embodiments, pneumatic compression may be used to provide therapy to a wearer's foot. FIG. 31A shows footwear 1100 according to some embodiments. Footwear 1100 has upper 1102 disposed on sole 1106. In some embodiments, upper 1102 may include zipper 1104A on the front of upper 1102. In some embodiments zipper 1004B may be located on the rear of upper 1102. In some embodiments, upper 1102 may be formed of one or more air bladders. Air bladders may be a component of a compression system that includes other components related to the inflation of air bladders. The air bladders may be filled by a pump 1112 disposed in sole 1106. Pump 1112 supplies pressurized air to valve block 1108. In some embodiments, pump 1112 is a pneumatic pump. Valve block 1108 includes several valves that are fluidly disposed between pump 1112 and air bladders. Valve 1108 is configured to permit the flow of pressurized air from pump 1112 to air bladders through tubes 1110. Footwear 1100 also includes module 1114. Module 1114 may include controllers, batteries, PCB modules, or other accessories. The air bladders apply pressure to the foot of the wearer by inflating. The pressure may be varied or constant.

FIG. 31B shows another embodiment of footwear 1200 with air bladders 1206. Footwear 1200 has upper 1202 and sole 1204. Upper 1202 includes five air bladders 1206A-E. Air bladders 1206 may be arranged to act as a pneumatic compression layer by stacking multiple air bladders over an area. In some embodiments, air bladders 1206 are contained in ridges formed in the upper 1202 and covering the toes of the wearer's foot. Air bladders 1206A-E may inflate sequentially (i.e. A, then B, then C, then D, then E, or E, then D, then C, then B, then A). Each air bladder 1206 includes air bladder valves 1208A-E. In some embodiments, one or more air bladders 1206 are connected to the same air bladder valve 1208. Air bladders 1206 on the same side of upper 1202 (either left or right) may be connected to the same air bladder valves 1208. Air bladder valves 1208 may be accessible and operable by the wearer. For example, the wearer may be able to press on air bladder valves 1208 to release air from air bladders 1206. Air bladder valves 1208 may be disposed behind fabric layers that hide air bladder valves 1208 from view. In some embodiments, air bladder valves 1208 are connected by tubes 1212 to air pump 1210. Air pump 1210 is configured to supply pressurized air to air bladder valves 1208 to fill air bladders 1206. Air bladder valves 1208 may also be operated by a controller. The controller may adjust the level of inflation of air bladders 1206. The controller may adjust the level of inflation of air bladders 1206 to provide a therapy regimen to a foot of the patient. Where air bladders 1206 are fluidly connected to each other, the controller may open or close valves disposed between air bladders 1206 to move between air bladders 1206. In some embodiments, air bladders 1206 are connected by a channel that goes around the zipper 1104.

FIG. 31C shows an embodiment of footwear 1300 according to some embodiments. Like the embodiment of footwear 1200 shown in FIG. 31B, footwear 1300 has air bladders 1306 and sole 1304. In this embodiment, sole 1304 is also equipped with an air bladder 1308 to provide pressure to the bottom of a wearer's foot.

FIG. 31D shows an embodiment of footwear 1400 according to some embodiments. Like the embodiments shown in FIGS. 31B and 31C, the embodiment of footwear 1400 shown in FIG. 31D includes zipper 1402 that divides airbladders 1406. The embodiment shown in FIG. 31D includes a cooling system 1408 disposed in sole 1404. Cooling system 1408 may be any number of cooling systems described herein, such as, for example, TEC cooling systems. Cooling system 1408 may cool all or most of a wearer's foot's sole, as shown in the embodiment in FIG. 31D, or cooling system 1408 may only cool a portion of a wearer's foot. For example, cooling system 1408 may be configured to cool only the heel of the wearer. In some embodiments, cooling system 1408 may be configured to activate only portions of the cooling system. Thus, cooling system 1408 can cover the entire sole 1404 but apply targeted cooling to the foot of the wearer as needed.

The article of footwear may include a compression system with a built-in sleeve that is configured to apply pressure to the wearer's foot. In some embodiments, the built-in sleeve is similar to footwear 1100, 1200, 1300 described above, except that it lacks a sole. In some embodiments, the compression system comprises a memory shape material, for example, nitinol. The compression system may also deform in response to heat to apply pressure to the foot of the wearer. The heat may be supplied either by a heat source disposed in the footwear or even by the foot of the wearer.

FIG. 31E shows an embodiment of footwear 1450 having upper 1452 and sole 1454. Upper 1452 and sole 1454 include vibration motors 1456. Vibration motors 1456 are configured to deliver vibrations to the wearer's foot. For example, the plurality of vibration motors 1456 is disposed in upper 1456 and is configured to deliver vibrations to the Achilles tendon and the top of the wearer's foot. The plurality of vibration motors 1456 are also configured to deliver vibrations to the bottom of the wearer's foot. Upper 1452 may also include zipper 1460. In some embodiments, air bladders 1458 disposed in upper 1452 are configured to provide compression while vibrations motors 1456 are operating. In some embodiments, sole 1454 has a cooling system 1462 configured to cool the foot of the wearer.

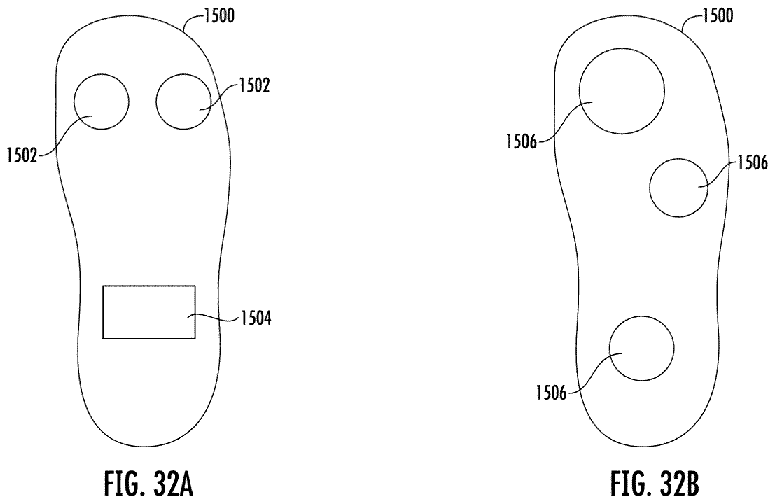

FIGS. 32A and 32B show a schematic view of a sole 1500 according to some embodiments. Sole 1500 is fitted with sensors. Sensors may be any one of pressure sensors, temperature sensors, heart rate sensors, moisture sensors, or any other suitable or needed sensor. Sensors can be used to monitor wearers for general healthcare tracking, to adjust treatments applied by an article of footwear, or to monitor compliance with medical treatments.

For example, pressure sensors 1502 may be located in sole 1500 of the footwear to determine whether a wearer of the footwear is sitting or standing. The intensity or frequency of the treatment can be altered according to whether the wearer is sitting or standing. Pressure sensors 1502 can also be used for determining whether a wearer is complying with medical treatment (e.g., diabetic neuropathy), as well as general healthcare tracking. As another example, temperature sensor 1504 can be used to optimize heating and cooling modalities in the footwear. The temperature sensor can also be used for healthcare tracking purposes. Heart rate sensors 1506 can be added in one or more locations to monitor heart rate. The heart rate sensor can be located on a top of the foot to measure a pedal pulse, or on the wearer's ankle to measure a posterior tibial pulse. The intensity or frequency of a treatment can be altered when a sleeping heart rate is detected.

Further implementations are within the CLAIMS. It will be understood that implementations of articles of footwear having one or more therapeutic assemblies include but are not limited to the specific components disclosed herein, as virtually any components consistent with the intended operation of various articles of footwear having one or more therapeutic assemblies may be utilized. Accordingly, for example, it should be understood that, while the drawings and accompanying text show and describe particular implementations of articles of footwear having one or more therapeutic assemblies, any such implementation may comprise any shape, size, style, type, model, version, class, grade, measurement, concentration, material, weight, quantity, and/or the like consistent with the intended operation of articles of footwear having one or more therapeutic assemblies.

The concepts disclosed herein are not limited to the specific articles of footwear having one or more therapeutic assemblies shown herein. For example, it is specifically contemplated that the components included in particular articles of footwear having one or more therapeutic assemblies may be formed of any of many different types of materials or combinations that can readily be formed into shaped objects and that are consistent with the intended operation of the articles of footwear having one or more therapeutic assemblies. For example, the components may be formed of: rubbers (synthetic and/or natural) and/or other like materials; glasses (such as fiberglass), carbon-fiber, aramid-fiber, any combination therefore, and/or other like materials; elastomers and/or other like materials; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; metals, such as zinc, magnesium, titanium, copper, iron, steel, carbon steel, alloy steel, tool steel, stainless steel, spring steel, aluminum, and/or other like materials; and/or any combination of the foregoing.

Furthermore, articles of footwear having one or more therapeutic assemblies may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular implementations of articles of footwear having one or more therapeutic assemblies, it should be readily apparent that a number of modifications may be made without departing from the spirit thereof and that these implementations may be applied to other implementations disclosed or undisclosed. The presently disclosed articles of footwear having one or more therapeutic assemblies are, therefore, to be considered in all respects as illustrative and not restrictive.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

1-51. (canceled)

52. An article of footwear for warm-up and active recovery, the article of footwear comprising:
a sole; and
an upper having a far infrared radiation heating element,
wherein the far infrared radiation heating element is configured to apply a therapeutic regimen.

53. The article of footwear of claim 52, wherein the far infrared radiation heating element comprises one of carbon fiber, graphene, and ceramics.

54. The article of footwear of claim 52, further comprising cold elements coupled to the sole.

55. The article of footwear of claim 54, wherein the cold elements and the far infrared radiation heating element are configured to operate sequentially to apply the therapeutic regimen.

56. The article of footwear of claim 54, wherein the cold elements and the far infrared radiation heating element are configured to not operate simultaneously.

57. The article of footwear of claim 54, wherein the cold elements comprise thermoelectric coolers.

58. The article of footwear of claim 54, wherein the cold elements comprise contact areas.

59-105. (canceled)

106. An article of footwear comprising:
a sole;
a far infrared radiation heating system comprising a body and two terminals configured to pass a current through the body, wherein the far infrared radiation heating system is configured to provide heat to a foot of a wearer of the article of footwear; and a thermoelectric cooler assembly configured to transfer heat away from the foot of the wearer.

107. The article of footwear of claim 106, wherein the body comprises one of carbon fiber, graphene, and ceramic.

108. The article of footwear of claim 106, wherein the body is formed of a mesh.

109. The article of footwear of claim 108, wherein the mesh comprises carbon fiber strands.

110. The article of footwear of claim 106, wherein the far infrared radiation heating system is disposed in the sole.

111. The article of footwear of claim 106, wherein the thermoelectric cooler assembly is disposed in the sole.

112. The article of footwear of claim 106, further comprising an upper, wherein the far infrared radiation heating system is disposed in the upper.

113. The article of footwear of claim 106, further comprising:
an additional far infrared radiation heating system; and
an additional thermoelectric cooler assembly,
wherein the far infrared radiation heating system, the additional far infrared radiation heating system, the thermoelectric cooler assembly, and the additional thermoelectric cooler assembly are disposed in the sole.

114. An article of footwear comprising:
a sole having a far infrared heating system and a thermoelectric cooler assembly;
an upper having one or more far infrared heating systems; and
a controller electrically and operatively coupled to the far infrared heating system of the sole, the thermoelectric cooler assembly, and the one or more far infrared heating systems of the upper.

115. The article of footwear of claim 114, wherein the far infrared heating system of the sole is disposed in a forefoot region of the sole.

116. The article of footwear of claim 114, wherein the one or more far infrared heating systems of the upper extend from a medial side to a lateral side of the upper.

117. The article of footwear of claim 114, wherein the one or more far infrared heating systems of the upper comprises three or more far infrared heating systems disposed on a medial side of the upper and three or more far infrared heating systems disposed on a lateral side of the upper.

118. The article of footwear of claim 114, further comprising an air intake configured to draw in air to flow across a portion of the thermoelectric cooling system of the sole.
